(19) United States
(12) Patent Application Publication
(10) Pub. No.: US 2026/0202406 A1
(43) Pub. Date: Jul. 16, 2026

(54) METHODS OF PREDICTING AND TREATING IMMUNOTHERAPY ADVERSE EVENTS BASED ON IMMUNE CELL POPULATIONS

(71) Applicant: THE BOARD OF REGENTS OF THE UNIVERSITY OF TEXAS SYSTEM, Austin, TX (US)

(72) Inventors: David E. GERBER, Dallas, TX (US); John David FARRAR, Dallas, TX (US); Hong MU-MOSLEY, Dallas, TX (US); Jason Y. PARK, Dallas, TX (US)

(73) Assignee: THE BOARD OF REGENTS OF THE UNIVERSITY OF TEXAS SYSTEM, Austin, TX (US)

(21) Appl. No.: 19/135,568

(22) PCT Filed: Dec. 7, 2023
(86) PCT No.: PCT/US2023/082863

§ 371 Date: Jun. 4, 2025

Related U.S. Application Data
(60) Provisional application No. 63/386,387, filed on Dec. 7, 2022.

(51) Int. Cl. G01N 33/569; C12Q 1/6883; G01N 33/564; G01N 33/68

(52) U.S. Cl. CPC G01N 33/56972; C12Q 1/6883; G01N 33/564; G01N 33/6854; G01N 33/6866; G01N 33/6869; G01N 33/6887; C12Q 2600/106; G01N 2333/4709; G01N 2333/4712; G01N 2333/522; G01N 2333/523; G01N 2333/525; G01N 2333/5412; G01N 2333/545; G01N 2333/57; G01N 2333/72; G01N 2333/78

(57) ABSTRACT

The present disclosure generally relates to compositions and methods for predicting or diagnosing immune-related adverse events (irAE) before, during, or after immune checkpoint inhibitor (ICI) treatment in a subject with cancer. The method includes assessment of abundance or expression of immune cells, autoantibodies, and/or cytokines.

### CROSS-REFERENCE TO RELATED APPLICATION

This application claims priority to U.S. Provisional Patent Application Ser. No. 63/386,387, filed Dec. 7, 2022, and titled “METHODS OF PREDICTING AND TREATING IMMUNOTHERAPY ADVERSE EVENTS BASED ON IMMUNE CELL POPULATIONS”, which is incorporated by reference herein in its entirety.

### ACKNOWLEDGEMENT OF GOVERNMENT SUPPORT

This invention was made with support under Grant Nos. A1156189 and CA201543 awarded by the National Institutes of Health. The government has certain rights in the invention.

### BACKGROUND

### 1. Field

The present invention relates to identification of biomarkers for predicting, diagnosing, or monitoring immune-related adverse events associated with immune checkpoint inhibitor therapy.

### 2. Background

While immune checkpoint inhibitors (ICI) have revolutionized the treatment of cancer, associated toxicities termed immune-related adverse events (irAE) occur in a substantial proportion of patients. These autoimmune events may affect almost any organ system. Although irAE tend to occur within the first 3 months of ICI therapy, they may occur at almost any point during treatment. When irAE occur, the dose and duration of corticosteroids and other immunosuppressants remain somewhat arbitrary. Thus, there is a need for serial assessment of humoral and cellular immune parameters which can identify potential explanations for irAE timing as well as biologic effects of steroids, and provide humoral and cellular immune biomarkers across which may inform toxicity monitoring and management SUMMARY

In one aspect, the present disclosure provides a method of predicting the risk of developing and/or diagnosing immune-related adverse events (irAE) associated with immune checkpoint inhibitor (ICI) treatment in a subject. The method comprises (a) providing a sample from the subject; (b) assessing immune cell subset in the sample, wherein the assessment comprises determining the abundance of one or more immune cell subsets in the sample; and (c) predicting risk for developing/diagnosing irAE in the subject. In some aspects, the subject is predicted as having a high risk of developing or diagnosed with having irAE if the abundance of one or more of immune cell subsets in Table 1 (CD57hi CD56−TE CD8 T cells, CD57lo CD56−TE CD8 T cells, naïve B cells, naïve CD4 T cells, CD57hi CD56+TE CD8 T cells, memory B cells, EM CD8 T cells, CD57lo CD56+TE CD8 T cell, naïve CD8 T cells, gamma delta T cells, pDC, plasmablasts, CTLA-4+ naïve B cells, or any combination thereof) are different in the sample from the subject when compared to the abundance of the one or more of the immune cell subsets in a control sample.

In some aspects, the present disclosure comprises a method of monitoring the risk of developing irAE associated with ICI treatment in a subject. The method comprises (a) providing a sample from the subject; (b) assessing immune cell subset in the sample, wherein the assessment comprises determining the abundance of one or more of immune cell subsets in the sample; and (c) monitoring risk for developing/diagnosing irAE in the subject. In some aspects, the subject is predicted as having a high risk of developing irAE if the abundance of one or more of immune cell subsets in Table 1 (CD57hi CD56−TE CD8 T cells, CD57lo CD56−TE CD8 T cells, naïve B cells, naïve CD4 T cells, CD57hi CD56+TE CD8 T cells, memory B cells, EM CD8 T cells, CD57lo CD56+TE CD8 T cells, naïve CD8 T cells, gamma delta T cells, pDC, plasmablasts, CTLA-4+ naïve B cells, or any combination thereof) are different in the subject when compared to the abundance in a control sample.

In some aspects, the irAE comprises ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof. In some aspects, the assessment of immune cells is performed before ICI treatment to predict the subject's risk of developing irAE. In some aspects, the assessment of immune cells is performed during ICI treatment to diagnose the subject's risk of developing irAE. In some aspects, the assessment is performed after ICI treatment to monitor the subject's risk for developing irAE or the subject's recovery from irAE.

In some aspects, the subject is predicted as high risk of developing irAE if the abundance of one or more of naïve B cells, CD57hiCD56+TE CD8 T cells, memory B cells, gamma delta T cells, naïve CD8 T cells, naïve CD4 T cells, or any combination thereof prior to ICI treatment (baseline) or during ICI treatment are lower in the sample from the subject compared to the abundance in a control sample.

In some aspects, the subject is predicted as high risk of developing irAE if the abundance of one or more of CD57lo CD56− terminal effector (TE) CD8 cells, CD57hi CD56-CD16+TE CD8 T cells, CD57lo CD56+TE CD8 T cells, effector memory (EM) CD8 T cells, or any combination thereof, prior to ICI treatment (baseline) are elevated compared to the abundance in a control sample. In some aspects, the subject is diagnosed with irAE if the abundance of one or more of CD57lo CD56− terminal effector CD8 cells, CD57hi CD56− terminal effector CD8 T cells, EM CD8 T cells, plasmacytoid dendritic cells (pDCs), plasmablasts, CLTA-4+naïve B cells, or any combination thereof are elevated during ICI treatment, compared to the abundance in a control sample.

In some aspects, the sample is a whole blood, serum, plasma, cerebrospinal fluid, pleural fluid, pericardial fluid, peritoneal fluid, bone marrow, or tissue, urine, cerebrospinal fluid (CSF), or other body fluid. In some aspects, the ICI treatment is administered as part of a cancer treatment. In some aspects, the ICI treatment comprises administration of an inhibitor of PD-1, PD-L1, TIM-3, LAG-3, CTLA-4, CSF-1R, or any combination thereof.

In some aspects, assessing the immune cells comprises mass cytometry (cytometry by time-of-flight CyTOF), immune profiling assay, flow cytometry, fluorescence-activated cell sorting (FACS), or immunomagnetic separation.

In some aspects, the method further comprises assessing the expression of an autoantibody in the subject, wherein the autoantibody comprises one or more of angiotensin receptor (AGTR), α-actin, U1-snRNP A, amyloid, collagen 1l, or any combination thereof. In some aspects, the assessment comprises identifying if the expression of one or more autoantibodies are elevated in the sample from the subject compared to the expression in a control sample.

In some aspects, the method further comprises assessing the expression of a cytokine in the subject, wherein the cytokine is selected from one or more of IL-6, IL-1P, TNFα, IFNγ, CXCL2, CCL17, or any combination thereof. In some aspects, the assessment comprises identifying if the expression of one or more cytokines are elevated in the sample from the subject compared to the expression in a control sample.

In some aspects, the method further comprises repeating steps (a)-(c) at a second time point, thereby permitting determination of a change in the subject's risk of developing irAE and/or diagnosis of irAE in the subject.

In some aspects, the method further comprises further comprising predicting the subject as having low risk if (i) the abundance of one or more of naïve B cells, CD57hiCD56+TE CD8 T cells, memory B cells, gamma delta T cells, naïve CD8 T cells, naïve CD4 T cells, or any combination thereof prior to ICI treatment (baseline) or during ICI treatment are higher in the sample from the subject compared to the abundance in a control sample; (ii) the abundance of one or more of CD57lo CD56− TE CD8 cells, CD57hi CD56− TE CD8 T cells, effector memory (EM) CD8 T cells, CD57lo CD56+TE CD8 T cells, or combination thereof prior to ICI treatment (baseline) are lower or equivalent in the sample from the subject when compared to the abundance in a control sample; and/or (ii) if the abundance of one or more of CD57lo CD56− TE CD8 cells, CD57hiCD56− TE CD8 T cells, EM CD8 T cells, pDCs, plasmablasts, CLTA-4+ naïve B cells, or any combination thereof are lower or equivalent during ICI treatment, in the sample from the subject compared to the abundance in a control sample.

In some aspects, the method further comprises treating the subjectwith an ICI therapy when the subject is predicted to have a low risk for developing irAE. In some aspects, the method further comprises treating the subject predicted as having a high risk of developing irAE with a non-ICI therapy or treating the subject with an ICI therapy and an irAE mitigating therapy, wherein the irAE mitigating therapy comprises corticosteroids (e.g., prednisone, methylprednisolone, dexamethasone, budesonide), TNF inhibitors (e.g., infliximab), hormone replacement (e.g., hydrocortisone, levothyroxine), BRAF inhibitor (e.g. vemurafenib, dabrafenib, and encorafenib), MEK inhibitor (e.g. binimetinib, cobimetinib, selumetinib, trametinib), CXCL8 inhibitors (e.g. repertaxin), or any combination thereof.

In some aspects, the present disclosure provides a method of treating a subject with cancer comprising: (a) providing a sample from the subject; (b) assessing immune cell subsets in the sample, wherein the assessment comprises determining the abundance of one or more of immune cell subsets in Table 1 (CD57hi CD56− TE CD8 T cells, CD57lo CD56− TE CD8 T cells, naïve B cells, naïve CD4 T cells, CD57hi CD56+TE CD8 T cells, memory B cells, EM CD8 T cells, CD57lo CD56+TE CD8 T cell, naïve CD8 T cells, gamma delta T cells, pDC, plasmablasts, CTLA-4+ naïve B cells, or any combination thereof) in the sample; (c) predicting the subject's risk of developing irAE. In some aspects, wherein the subject is diagnosed as: (i) high risk when the abundance of one or more of naïve B cells, CD57hiCD56+TE CD8 T cells, memory B cells, gamma delta T cells, naïve CD8 T cells, naïve CD4 T cells, or any combination thereof are lower in the sample from the subject compared to the abundance in a control sample; (ii) low risk of developing irAE if the abundance of one or more of naïve B cells, CD57hiCD56+TE CD8 T cells, memory B cells, gamma delta T cells, naïve CD8 T cells, naïve CD4 T cells, or any combination thereof are higher in the sample from the subject compared to the abundance in a control sample; (iii) low risk when the abundance of one or more of CD57lo CD56− terminal effector CD8 cells, CD57hi CD56− terminal effector CD8 T cells, EM CD8 T cells, plasmacytoid dendritic cells, plasmablasts, CLTA-4+ naïve B cells, or any combination thereof are lower or equal in the sample from the subject when compared to the abundance in a control sample; or (iv) high risk when the abundance of one or more of CD57lo CD56− terminal effector CD8 cells, CD57hi CD56-terminal effector CD8 T cells, EM CD8 T cells, plasmacytoid dendritic cells, plasmablasts, and/or CLTA-4+naïve B cells are greater in the sample from the subject when compared to the abundance in a control sample. The method further comprises (d) treating the subject with: (i) an ICI therapy if subject is diagnosed as low risk of developing irAE; (ii) a non-ICI therapy if the subject is diagnosed as high risk of developing irAE; or (iii) an ICI therapy and an irAE mitigating therapy if the subject is diagnosed as high risk of developing irAE.

In some aspects, irAE comprises ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof.

In some aspects, the method further comprises detecting the expression of a cytokine in the sample wherein the cytokine comprises one or more of IL-6, IL-13, TNFα, IFNγ, CXCL2, CCL17, or any combination thereof. In some aspects, the method comprises identifying if expression of one or more of the cytokines are elevated in the sample from the subject compared to the expression in a control sample.

In some aspects, the method further comprises detecting the expression of an autoantibody in the sample wherein the autoantibody comprises one or more of angiotensin receptor (AGTR), α-actin, U1-snRNP A, amyloid, collagen 1l, or any combination thereof. In some aspects, the method comprises identifying if expression of one or more of autoantibodies are elevated in the sample from the subject compared to the expression in a control sample.

In some aspects, the said ICI therapy comprises administration of an inhibitor of PD-1, PD-L1, TIM-3, LAG-3, CTLA-4, CSF-1R, or any combination thereof.

In a further aspect, the disclosure provides a method of identifying the presence of at least one differentially abundant immune cell subset associated with irAE in a biological sample of a subject with cancer, the method comprising: providing a sample from the subject; assessing immune cells in the sample, wherein the assessment comprises detecting the abundance of one or more of immune cell subsets disclosed in Table 1 (CD57hi CD56− TE CD8 T cells, CD57lo CD56− TE CD8 T cells, naïve B cells, naïve CD4 T cells, CD57hi CD56+TE CD8 T cells, memory B cells, EM CD8 T cells, CD57lo CD56+TE CD8 T cell, naïve CD8 T cells, gamma delta T cells, pDC, plasmablasts, CTLA-4+ naïve B cells, or combination thereof); and comparing the abundance of one or more immune cell subsets to the abundance in a control sample to identify the presence of differentially expressed immune cell subset(s).

In some aspects, the subject is planning, undergoing, or has completed immune checkpoint inhibitor (ICI) treatment. In some aspects, irAE comprises ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof.

In some aspects, the method further comprises detecting the expression of a cytokine in the sample wherein the cytokine comprises one or more of IL-6, IL-1R, TNFα, IFNγ, CXCL2, CCL17, or any combination thereof. In some aspects, the method comprises identifying if the expression of one or more of cytokines are elevated in the sample from the subject compared to the expression in a control sample.

In some aspects, the method further comprises detecting the expression of an autoantibody in the sample wherein the autoantibody comprises one or more of angiotensin receptor (AGTR), α-actin, U1-snRNP A, amyloid, collagen II, or any combination thereof. In some aspects, the method comprises identifying if the expression of one or more autoantibodies are elevated in the sample from the subject as compared to the expression in a control sample.

In some aspects, the assessment comprises determining a baseline or a pre-treatment profile that correlates with future toxicity. In some aspects, the baseline or a pre-treatment profile comprises one or more of CD57lo CD56− terminal effector TE CD8 cells, CD57+CD16+CD57hi CD56− terminal effector CD8 T cells, Effector memory (EM) EM CD8 T cells, NK T plasmacytoid dendritic cells (pDCs), or any combination thereof and/or one or more of naïve B cells, CD57hiCD56+TE CD8 T cells, memory B cells, gamma delta T cells, naïve CD8 T cells, naïve CD4 T cells, or any combination thereof. In some aspects, the assessment comprises an interim profile that correlates with ICI treatment before the onset of irAE. In some aspects, the assessment comprises a profile that correlates with onset of irAE during treatment. In some aspects, the profile that correlates with onset of toxicity during treatment comprises one or more of CD57lo CD56− TE CD8 cells, CD57hi CD56− TE CD8 T cells, EM CD8 T cells, plasmacytoid dendritic cells (pDCs), plasmablasts, CLTA-4+ naïve B cells, or any combination thereof and/or one or more of naïve B cells, CD57hiCD56+TE CD8 T cells, memory B cells, gamma delta T cells, naïve CD8 T cells, naïve CD4 T cells, or any combination thereof.

In some aspects, the control sample is procured from a subject with low risk of developing irAE.

Other technical features may be readily apparent to one skilled in the art from the following figures, descriptions, and claims.

### BRIEF DESCRIPTION OF THE DRAWINGS

FIG. 1 is a scheme depicting the timeline of clinical events and biospecimen collection.

FIG. 2A-2D show heatmap of cytokines and antibodies. FIG. 2A-2B show heat map of cytokine across time-points with comparison to the no toxicity cases. FIG. 2C-2D show heat map of antibodies across time-points with comparison to the no toxicity cases. BL, baseline. Both full panels (upper) and elevated panels specific to the patient (bottom) are shown. Heatmaps from healthy controls have been previously published (Khan et al. Immune dysregulation in cancer patients developing immune-related adverse events. Br J Cancer. 2019; 120(1):63-8).

FIG. 3A-3F display immune cell profiles of the patient, no toxicity cases (NT), and healthy controls in peripheral blood by CyTOF. FIG. 3A shows a total 41 clusters were identified in all 23 files corresponding to B cells, CD4 T cells, CD8 T cells, monocytes, and NK cells. Colored dots in UMAP plot represent cell events. FIG. 3B shows heatmap of surface marker expression in each cluster on patient baseline sample. The median antigen expression is shown in the heatmap. FIG. 3C shows volcano plot shows clusters with significant differences between the patient, no toxicity cases (NT), and healthy controls (CTRL) by EdgR statistical analysis. Significant clusters are shown in green, q<0.01. FIG. 3D shows heatmap of clusters with changes among all time-points from patient, no toxicity cases, and healthy controls. The abundance of cell events per cluster are shown. FIG. 3E shows box chart showing differences in clusters between patients with cancer (with and without irAE) and healthy controls. FIG. 3F shows box chart showing differences between the patient and no toxicity cases+healthy controls. The percentage of the total event count per cluster is shown. Solid lines designate median; dotted lines designate mean. Bars represent maximum and minimum values. Dots adjacent to boxes represent individual samples.

FIG. 4 depicts immune cell profiles from the patient, no toxicity cases (NT), and healthy controls in peripheral blood by CyTOF. UMAP plots of the clusters with changes among all sample time-points for the patient, no toxicity cases, and healthy controls. Dots represent cell events.

FIG. 5 displays violin chart showing differences in immune cell subsets at baseline (BL) prior to ICI therapy between the patient irAE and the controls of NT cases and healthy subjects. The percentage of the total event count is shown. Solid lines designate median; dotted lines designate mean. Bars represent maximum and minimum values. Dots adjacent to boxes represent individual samples. Bars represent maximum and minimum values.

FIG. 6 depicts a method for immunophenotyping human peripheral blood mononuclear cells by mass cytometry.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various aspects of the present disclosure. The drawings and description are intended to describe aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

Provided herein are methods of predicting, diagnosing and/or monitoring immune-related adverse events (irAE) before, during or after immune checkpoint inhibitor (ICI) treatment in a subject. The present disclosure is partly based on the surprising determination that subjects develop a unique immune cell profile that correlates with irAE before, and/or during ICI treatment administered as part of cancer therapy.

### I. Terminology

For the purposes of promoting an understanding of the principles of the present disclosure, reference will now be made to preferred aspects and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combinations.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human. In other aspects, the subject can be a human in need of treating a cancer.

As used herein “immune-related adverse event” or “irAE” refers to diverse toxicities, side-effects or problems associated with cancer immunotherapy. Immunotherapy include therapy using immunotoxins, T-cell transfer, chimeric antigen receptors, antibodies, immune system modulators, immune checkpoint inhibitors, and/or other immunotherapies known to those of skill in the art.

In some aspects, as disclosed irAE can comprise ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof.

As used herein “immune checkpoint inhibitor” or “IC” refers to a drug that blocks immune checkpoints. These checkpoints are a normal part of the immune system and keep immune responses from being too strong. By blocking them, these drugs allow immune cells to respond more strongly to cancer. Immune checkpoint inhibitors work by preventing cancer cells from turning T-cells (white blood cells that detect infections and abnormalities) off. Non-limiting examples of immune checkpoint inhibitors include inhibitors of PD-1, PD-L1, TIM-3, LAG-3, CTLA-4, and CSF-1R and any combination thereof. The immune checkpoint receptors may be on tumor cells or immune cells such as T cells, monocytes, microglia, and macrophages, without limitation. The agents which assert immune checkpoint blockade may be small chemical entities or polymers, antibodies, antibody fragments, single chain antibodies or other antibody constructs, including, but not limited to, bispecific antibodies and diabodies. Immune checkpoint inhibitors which may be used according to the invention include any that disrupt the inhibitory interaction of cytotoxic T cells and tumor cells. These include but are not limited to anti-PD-1 antibody, anti-PD-L1 antibody, anti-CTLA4 antibody, anti-LAG-3 antibody, anti-TIM-3 antibody. The inhibitor need not be an antibody but can be a small molecule or other polymer. If the inhibitor is an antibody it can be a polyclonal, monoclonal, fragment, single chain, or other antibody variant construct. Inhibitors may target any immune checkpoint known in the art, including but not limited to, CTLA-4, PDL1, PDL2, PD1, B7-H3, B7-H4, BTLA, HVEM, TIM3, GAL9, LAG3, CSF-1R, VISTA, KIR, 2B4, CD160, CGEN-15049, CHK1, CHK2, A2aR, CD28, CD86 and the B-7 family of ligands. Combinations of inhibitors for a single target immune checkpoint or different inhibitors for different immune checkpoints may be used. Illustrative examples of immune checkpoint inhibitors include CTLA-4 blocking antibodies (ipilimumab (Yervoy™), tremelimumab (Imjuno™)), PD-1 inhibitors (pembrolizumab (Keytruda®), nivolumab (Opdivo^{T}M), cemiplimab (Libtayo™), CT-011 (Pidilizumab™), AMP224, PD-L1 inhibitors (atezolizumab (Tecentriq™), avelumab (Bavencio™) durvalumab (Imfinzi™), BMS-936559), Lag3 inhibitors (Relatlimab™), combination of Lag3 and PD1 inhibitor (PD-1 inhibitor nivolumab (Opdualag™) OX40 inhibitor (MED16469), CD160 inhibitor (BY55). Non-limiting examples of inhibitors of CSF-1R include PLX3397, PLX486, RG7155, AMG820, ARRY-382, FPA008, IMC-CS4, JNJ-40346527, and MCS 110. The terms “ICI treatment”, “ICI therapy”, “ICI compounds”, and the like, refer to one or more ICI (or the use thereof) disclosed herein or known to those of skill in the art.

As used herein “ocular toxicity” refers to one or more adverse changes that affect the structure or function of the eye. Ocular toxicity can manifest as a gross and/or histologic retinal and/or vitreous toxic reaction. Evidence of such a toxic reaction may include one or more of white vitreous bodies, white vitreous opacities, electroretinography abnormalities such as reduction in mean B-wave amplitude in both scotopic and photopic conditions, occlusion of the temporal retinal vessels, and fibrin deposits. Ocular toxicity can also be uveitis, dry eye syndrome, ocular myasthenia, myositis, ophthalmoplegia, ptosis, Sjogren's, metastases to the vitreous, vasculitis, damage to rod/cone function, macular edema, retinopathy, choroidal neovascularization, chorioretinal atrophy, vascular leakage, choroidal sarcoid granulomas, choroidal detachment, development of subretinal fluid, optic neuritis, nerve palsies associated with eye, arteritic ischemic optic neuropathy, and/or eye inflammation.

As uses herein “autoantigen” refers to a normal protein or protein complex (and sometimes DNA or RNA) that is recognized by the immune system of patients suffering from a specific autoimmune disease. These antigens should not be, under normal conditions, the target of the immune system, but their associated T cells are not deleted and instead attack.

As used herein “cytokine” refers to a broad category of small proteins that are important in cell signaling. Release of cytokine has an effect on the behavior of cells around them. Cytokines are involved in autocrine signaling, paracrine signaling and endocrine signaling as immunomodulating molecules. Non-limiting examples of cytokines include chemokines, interferons, interleukins, lymphokines, and tumor necrosis factors. Cytokines are produced by a variety of cell types including immune cells like macrophages, monocytes, dendritic cells, B lymphocytes, T lymphocytes and mast cells, as well as endothelial cells, fibroblasts, and various stromal cells; and a given cytokine may be produced by more than one type of cell.

As used herein “immune cell” refers to a cell which develops from stem cells in the bone marrow and become different types of white blood cells. Immune cells include neutrophils, eosinophils, basophils, mast cells, monocytes, macrophages, dendritic cells, natural killer cells, and lymphocytes (B cells and T cells).

As used herein “abundance” refers to the amount of a particular analyte (e.g., immune cell subset) present in the sample. The amount may be a number, ratio, proportion, or a percentage of the analyte compared to the control sample. The amount may be an absolute amount or a relative amount.

As used herein “expression” or “expression level” or “level of expression” refers to amount of a particular analyte (e.g., antibody or cytokine) present in the sample. The amount may be a number, ratio, proportion, or a percentage of the analyte compared to the control sample. The amount may be an absolute amount or a relative amount.

As used herein “cancer” may be one or more neoplasm or cancer. The neoplasm may be malignant or benign, the cancer may be primary or metastatic; the neoplasm or cancer may be early stage or late stage. Non-limiting examples of neoplasms or cancers include acute lymphoblastic leukemia, acute myeloid leukemia, adrenocortical carcinoma, AIDS-related cancers, anal cancer, appendix cancer, astrocytoma (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (soft tissue, uterine), Sézary syndrome, skin cancers (nonmelanoma), skin carcinoma, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, and Wilms tumor (childhood).

In some aspects, treatment of cancer can comprise increased inhibition of cancer progression and/or metastases, inhibition of an increase in tumor volume, a reduction in tumor volume and/or growth, a reduction in tumor growth rate, an eradication of a tumor and/or cancer cell, or any combination thereof. In some aspects, the treatment can also prolong the survival of a subject, and/or improve the prognosis.

The biological sample may be of any biological tissue, fluid, or cell from the subject. The sample can be solid or fluid. The sample can be a heterogeneous cell population. Non-limiting examples of suitable biological samples include sputum, serum, blood, blood cells (e.g., white cells), a biopsy, urine, peritoneal fluid, pleural fluid, or cells derived therefrom. The biopsy can be a fine needle aspirate biopsy, a core needle biopsy, a vacuum assisted biopsy, an open surgical biopsy, a shave biopsy, a punch biopsy, an incisional biopsy, a curettage biopsy, or a deep shave biopsy. Biological samples may also include sections of tissues, such as frozen sections or formalin fixed sections taken for histological purposes. A sample can be a tumor tissue, tissue surrounding a tumor, or non-tumor tissue. Methods of collecting a biological sample from a subject are well known in the art. In some aspects, the biological sample is a peripheral blood sample. In some aspects, the biological sample is peripheral blood mononuclear cell (PBMC).

Sample from the subject can be procured one or more times, before, during and/or after diagnosis. In some aspects, samples can be procured from the subject before, during, and/or after treatment of cancer. In some aspects, sample can be procured from the subject before start of cancer treatment. In some aspects, sample can be procured from the subject undergoing ICI treatment, before onset of irAE. In other aspects, sample can be procured after onset of irAE in a subject. In some aspects, sample can be procured before, during and/or after administration of a non-ICI cancer treatment, or ICI treatment combined with steroid treatment, for monitoring the treatment for irAE. Additionally, samples can be procured repeatedly at multiple stages after initial sample procurement, to determine and/or monitor irAE in a subject.

In some aspects, the sample can be obtained from the subject prior to the initiation of ICI treatment. In some aspects, the sample can be obtained from the subject about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, or more after the initiation of the ICI treatment. In some aspects, the sample can be obtained at about 2-4 weeks after the initiation of the ICI treatment. In some aspects, the sample is obtained at about 6-8 weeks after the initiation of the ICI treatment. In some aspects, samples can be obtained before initiation of ICI treatment, two weeks, six weeks, three months, after ICI treatment, and/or every three months after initiation of steroids for irAE treatment.

In some aspects, control sample can be procured from a healthy subject and/or a subject undergoing ICI treatment but that does not have irAE or ICI toxicity. In some aspects, the control sample can comprise of non-cancer cells. In some aspects, the non-cancer cells can be from the same tissue type as the cancer cells. For example, if the cancer cells are from breast cancer, then the non-cancer cells can be from healthy breast tissue. In some aspects, the control can comprise of an average levels of the biomarker profile in a sample from a subject before onset of cancer. In some aspects, control sample can be a sample from the subject prior to diagnosis or treatment. In certain aspects, the biomarker profile can be measured in a person or persons other than the subject with cancer. In some aspects, the control a person or persons with similar characteristics to the subject with cancer. In some aspects, the control can be an average of the combination of disclosed biomarker levels from different healthy sources (e.g., more than one healthy control subject and/or more than one subject that does not have irAE). In some aspects, the control sample can be pooled sample. In some aspects, the control sample is procured from a subject with low risk of developing irAE.

As used herein, a subject that has a low risk of developing irAE can be a subject or population that does not develop irAE with ICI treatment. In an aspect, a subject that has a low risk of developing irAE can be a subject or population that does not develop irAE with ICI treatment as determined through retrospective analysis to not develop irAE with ICI treatment.

In some aspects, the assessment of abundance, level, or expression of one or more immune cell subtypes, autoantibodies and/or cytokines comprises comparing the abundance, level, or expression of one or more immune cell subtypes, autoantibodies and/or cytokines in the sample from the subject to the abundance, level, or expression of the same one or more immune cell subtypes, autoantibodies and/or cytokines in a control sample

### II. Biomarker Profiles and Methods of Predicting Risk and/or Diagnosing IRAE

The present disclosure provides immunological characteristics of ICI-related irAE and insights into the biological profiles of ICI-related irAE. These profiles can be used as biomarkers to provide rationales for potential cancer treatment options.

### Immune Cell Profile

In some aspects, the present disclosure provides immune cell profiles related to ICI-associated irAE. In some aspects, the immune cell profile comprises one or more of immune cell subsets that are different in abundance in a subject with irAE when compared to the abundance in a control sample. In some aspects, the immune cell profile comprises one or more of the immune cell subsets disclosed in Table 1. In some aspects, the immune cell profile comprise one or more of baseline or pre-treatment immune cell subsets at an abundance that correlate with future irAE. In some aspects, the immune cell profile comprises an abundance of interim immune cell subsets, obtained during ICI treatment before the onset of irAE. In some aspects, the immune cell profile comprises an immune cell subset abundance that correlates with onset of irAE during treatment. In some aspects, the immune cell subsets are used as biomarkers to assess the risk of a subject developing irAE before, during, or after ICI treatment. In some aspects, the elevated abundance of one or more immune cell subset is reduced in the subject after treatment with irAE mitigating therapy.

The immune cell subsets disclosed herein, may include those in Table 1. One or more of the immune cell subsets of Table 1 may be utilized to assess the risk of a subject developing irAE before, during, or after ICI treatment.

TABLE 1
Immune Cell Subsets

| Subset | Immune Cell Phenotype |
|---|---|
| 1 | CD57hi CD56− TE CD8 T cells |
| 2 | CD57lo CD56− TE CD8 T cells |
| 3 | naïve B cells |
| 4 | naïve CD4 T cells |
| 5 | CD57hi CD56+ TE CD8 T cells |
| 6 | memory B cells |
| 7 | EM CD8 T cells |
| 8 | CD57lo CD56+ TE CD8 T cell |
| 9 | naïve CD8 T cells |
| 10 | gamma delta T cells |
| 11 | pDC |
| 12 | plasmablasts |
| 13 | CTLA-4+ naïve B cells |

In some aspects, immune cell subset 1 comprises immune cells that express the antigen markers at high levels: CD57 (e.g., CD57 hi), express antigen markers: CD3, CD8, and CD45RA (e.g., CD3+, CD8+, and CD45RA+) and that do not express the antigen markers: CCR7, CD45RO and CD56 (e.g., CCR7−CD45RO− and CD56−).

In some aspects, immune cell subset 2 comprises immune cells that express the antigen markers: CD3, CD8, and CD45RA (e.g., CD3+, CD8+, and CD45RA+), express at low levels antigen marker: CD57 (e.g., CD57lo) and that do not express the antigen markers: CCR7, CD45RO, CD57 and CD56 (e.g., CCR7−, CD45RO−, CD57−, and CD56−).

In some aspects, immune cell subset 3 comprises immune cells that express at high level the antigen markers: IgD (e.g., IgDhi), that express antigen markers: CD19 and CD20 (e.g., CD19+ and CD20+) and that do not express the antigen markers: CD3 and CD27 (e.g., CD3− and CD27−).

In some aspects, immune cell subset 4 comprises immune cells that express the antigen markers: CD3, CD4, CCR7, and CD45RA (e.g., CD3+, CD4+, CCR7+, and CD45RA+) and that do not express the antigen markers: CD45RO (e.g., CD45RO−)

In some aspects, immune cell subset 5 comprises immune cells that express at high levels the antigen markers: CD57 (e.g., CD57 hi), that express antigen markers: CD3, CD8, CD45RA, CD56 (e.g., CD3+, CD8+, CD45RA+, and CD56+) and that do not express the antigen markers: CCR7, CD45RO, and CD161 (e.g., CCR7−, CD45RO−, and CD161−)

In some aspects, immune cell subset 6 comprises immune cells that express the antigen markers: CD19 and CD20 (e.g., CD19+, and CD20+), express at low level the antigen markers: IgD, and CD27 (e.g., IgDlo, and CD2710) and that do not express antigen marker: CD3 (e.g., CD3−)

In some aspects, immune cell subset 7 comprises immune cells that express the antigen markers: CD3, CD8, and CD45RO (e.g., CD3+CD8+, and CD45RO) and that do not express the antigen markers: CCR7, and CD45RA (e.g., CCR7−, and CD45RA−).

In some aspects, immune cell subset 8 comprises immune cells that express the antigen markers: CD3, CD8, CD56, and CD45RA (e.g., CD3+, CD8+, CD56+, and CD45RA+), that express at low levels antigen markers: CD57 (e.g., CD57lo) and that do not express the antigen markers: CCR7, CD45RO, CD161 (e.g., CCR7−, CD45RO−, CD161−).

In some aspects, immune cell subset 9 comprises immune cells that express the antigen markers: CD3, CD8, CCR7, CD45RA (e.g., CD3+, CD8+, CCR7+, CD45RA+) and that do not express the antigen marker: CD45RO (e.g., CD45RO−).

In some aspects, immune cell subset 10 comprises immune cells that express the antigen markers: CD3, TCRgd, CD45RO, and CD27 (e.g., CD3+, TCRgd+, CD45RO+, and CD27+) and that do not express the antigen markers: CD4, and CD8 (e.g., CD4−, and CD8−).

In some aspects, immune cell subset 11 comprises immune cells that express the antigen markers: CD14, HLA-DR+, CD123, and CXCR3 (e.g., CD14+, HLA-DR+, CD123, and CXCR3+) that express at low level antigen marker: CD45 (e.g., CD451o) and that do not express the antigen markers: CD3, CD19, and CD20 (e.g., CD3−, CD19−, and CD20−).

In some aspects, immune cell subset 12 comprises immune cells that express the antigen markers: CD19, CD38 (e.g., CD19+, CD38+), and that do not express the antigen markers: CD3, and CD20, (e.g., CD3−, and CD20−)

In some aspects, immune cell subset 13 comprises immune cells that express the antigen markers: CD19, CD20, IgD, and CTLA4 (e.g., CD19+, CD20+, IgD+, and CTLA4+) and that do not express the antigen markers: CD3, and CD27 (e.g., CD3−, and CD27−)

In some aspects, the present disclosure provides method of predicting the risk of developing and/or diagnosing immune-related adverse events (irAE) associated with immune checkpoint inhibitor (ICI) treatment in a subject. The method comprises providing a sample from the subject, assessing one or more of immune cell subsets in the sample and predicting risk for developing/diagnosing irAE in the subject. The assessment of immune cells comprises determining the abundance of one or more immune cell subsets in the sample. The method further comprises predicting the subject as having a high risk of developing or diagnosed with having irAE if the abundance of one or more of immune cell subset in Table 1 are different in the subject when compared to the abundance in control sample.

In some aspects, the method comprises predicting the subject as having a high risk of developing or diagnosed with having irAE if the abundance of at least immune cell subsets in Table 1 are different in the sample from the subject when compared to the abundance in control sample. In some aspects, the method comprises predicting the subject as having a high risk of developing or diagnosed with having irAE if the abundance of at least two immune cell subsets in Table 1 are different in the sample from the subject when compared to the abundance in a control sample. In some aspects, the method comprises predicting the subject as having a high risk of developing or diagnosed with having irAE if the abundance of at least three immune cell subsets in Table 1 are different in the sample from the subject when compared to the abundance in a control sample. In some aspects, the method comprises predicting the subject as having a high risk of developing or diagnosed with having irAE if the abundance of at least four immune cell subsets in Table 1 are different in the sample from the subject when compared to the abundance in a control sample. In some aspects, the method comprises predicting the subject as having a high risk of developing or diagnosed with having irAE if the abundance of at least five immune cell subsets in Table 1 are different in the sample from the subject when compared to the abundance in a control sample. In some aspects, the method comprises predicting the subject as having a high risk of developing or diagnosed with having irAE if the abundance of at least six immune cell subsets in Table 1 are different in the sample from the subject when compared to the abundance in a control sample. In some aspects, the method comprises predicting the subject as having a high risk of developing or diagnosed with having irAE if the abundance of at least seven immune cell subsets in Table 1 are different in the sample from the subject when compared to the abundance in a control sample. In some aspects, the method comprises predicting the subject as having a high risk of developing or diagnosed with having irAE if the abundance of at least eight immune cell subsets in Table 1 are different in the sample from the subject when compared to the abundance in a control sample. In some aspects, the method comprises predicting the subject as having a high risk of developing or diagnosed with having irAE if the abundance of at least nine immune cell subsets in Table 1 are different in the sample from the subject when compared to the abundance in a control sample. In some aspects, the method comprises predicting the subject as having a high risk of developing or diagnosed with having irAE if the abundance of at least ten immune cell subsets in Table 1 are different in the sample from the subject when compared to the abundance in a control sample. In some aspects, the method comprises predicting the subject as having a high risk of developing or diagnosed with having irAE if the abundance of at least eleven immune cell subsets in Table 1 are different in the sample from the subject when compared to the abundance in a control sample. In some aspects, the method comprises predicting the subject as having a high risk of developing or diagnosed with having irAE if the abundance of at least twelve immune cell subsets in Table 1 are different in the sample from the subject when compared to the abundance in a control sample. In some aspects, the method comprises predicting the subject as having a high risk of developing or diagnosed with having irAE if the abundance of at least thirteen immune cell subsets in Table 1 are different in the sample from the subject when compared to the abundance in a control sample.

In some aspects of the present disclosure, a subject can be predicted as having a higher risk for developing and/or diagnosed with irAE associated with immune checkpoint inhibitor (ICI) treatment, when the immune cell profile comprises one or more cell subsets at a lower abundance in the sample from the subject when compared to a control sample. In some aspects of the present disclosure, a subject can be predicted as having a higher risk for developing and/or diagnosed with irAE associated with immune checkpoint inhibitor (ICI) treatment, when the immune cell profile comprises one or more cell subsets at a lower abundance in the sample from the subject when compared to a control sample, before initiation of ICI treatment at baseline, or after ICI treatment. In some aspects, the one or more immune cell subsets related to irAE associated with ICI treatment that are present at lower abundance can be one or more of naïve B cells, CD57hiCD56+TE CD8 T cells, memory B cells, gamma delta T cells, naïve CD8 T cells, naïve CD4 T cells, or any combination thereof. In some aspects, the one or more immune cell subsets related to irAE associated with ICI treatment that are present at lower abundance before initiation of ICI treatment at baseline, or after ICI treatment is naïve B cells. In some aspects, the one or more immune cell subsets related to irAE associated with ICI treatment that are present at lower abundance before initiation of ICI treatment at baseline, or after ICI treatment is CD57hiCD56+TE CD8 T cells. In some aspects, the one or more immune cell subsets related to irAE associated with ICI treatment that are present at lower abundance before initiation of ICI treatment at baseline, or after ICI treatment is memory B cells. In some aspects, the one or more immune cell subsets related to irAE associated with ICI treatment that are present at lower abundance before initiation of ICI treatment at baseline, or after ICI treatment is a gamma delta T cell subset. In some aspects, the one or more immune cell subsets related to irAE associated with ICI treatment that are present at lower abundance before initiation of ICI treatment at baseline, or after ICI treatment is a naïve CD8 T cell subset. In some aspects, the one or more immune cell subsets related to irAE associated with ICI treatment that is present at lower abundance before initiation of ICI treatment at baseline, or after ICI treatment is a naïve CD4 T cell subset.

In some aspects, the abundance of immune cells in an immune cell subset (e.g., one or more of naïve B cells, CD57hiCD56+TE CD8 T cells, memory B cells, gamma delta T cells, naïve CD8 T cells, and/or naïve CD4 T cells) can be determined from a sample by determining the number of immune cells in the sample that fall into the subset. (For example, X of the immune cells in the sample are subset 1.) The number of immune cells in a given subset can then be compared to the number of immune cells in that subset in a control sample. In some aspects, during assessment, the abundance (e.g., number) of immune cells in the one or more immune cell subset can be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower in the sample from the subject as compared to the abundance of the immune cells in the one or more immune cell subset in a control sample. In some aspects, number of the immune cells in the one or more immune cell subset can be at least about 25% lower as compared to the number in a control sample.

In some aspects, the abundance of immune cells in an immune cell subset (e.g., one or more of naïve B cells, CD57hiCD56+TE CD8 T cells, memory B cells, gamma delta T cells, naïve CD8 T cells, naïve CD4 T cells) can be determined from a sample by determining the percentage of immune cells in the sample that fall into the subset. (For example, X % of the immune cells in the sample are subset 1.) The percentage of immune cells in a given subset can then be compared to the percentage of immune cells in that subset in a control sample. In some aspects, the abundance (e.g., percentage) of immune cells in the one or more immune cell subsets can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points lower in a sample from the subject as compared to the abundance of the immune cells in the one or more immune cell subset in a control sample. In some aspects, number of the immune cells in the one or more immune cell subset can be at least about 10 percentage points lower in a sample from the subject as compared to the number in a control sample.

In some aspects, a subject can be predicted as having a higher risk for developing and/or diagnosed with irAE associated with ICI treatment, when the immune cell profile comprises one or more immune cell subsets at a greater abundance in the sample from the subject compared to the abundance in a control sample, before the initiation of ICI treatment. In some aspects, the pre-treatment or baseline immune cell subsets when present at higher abundance that is associated with higher risk of developing irAE associated with ICI treatment can comprise one or more of TE CD8 T cells, effector memory (EM) CD8 T cells, CD57lo CD56+TE CD8 T cell, or any combination thereof. In some aspects, the pre-treatment or baseline immune cell subsets when present at higher abundance that is associated with higher risk of developing irAE associated with ICI treatment can comprise TE CD8 T cells. In some aspects, the TE CD8 T cells can comprise CD57− terminal effector CD8 cells, CD57hi CD56− CD8 T cells, or any combination thereof. In some aspects, the pre-treatment or baseline immune cell subsets when present at higher abundance that is associated with higher risk of developing irAE associated with ICI treatment can comprise effector memory (EM) CD8 T cells. In some aspects, the pre-treatment or baseline immune cell subsets when present at higher abundance that is associated with higher risk of developing irAE associated with ICI treatment can comprise CD57lo CD56+TE CD8 T cell.

In some aspects, the abundance of immune cells in an immune cell subset (e.g., one or more of TE CD8 T cells, effector memory (EM) CD8 T cells, CD57lo CD56+TE CD8 T cell, or any combination thereof) can be determined from a sample by determining the number of immune cells in the sample that fall into the subset. (For example, X number of the immune cells in the sample are subset I number of immune cells in a given subset in the sample can then be compared to the number of immune cells in that subset in a control sample. In some aspects, the abundance (e.g., number) of immune cells in the one or more immune cell subsets can be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in the sample as compared to a control sample. In some aspects, the number of immune cells in the one or more immune cell can be more than 100% in a sample from the subject when compared to a control sample.

In some aspects, the abundance of immune cells in an immune cell subset (e.g., one or more of TE CD8 T cells, effector memory (EM) CD8 T cells, CD57lo CD56+TE CD8 T cell, or any combination thereof) can be determined from a sample by determining the percentage of immune cells in the sample that fall into the subset. (For example, X % of the immune cells in the sample are subset 1.) The percentage of immune cells in a given subset can then be compared to the percentage of immune cells in that subset in a control sample. In some aspects, the abundance (e.g., percentage) of immune cells in the one or more immune cell subsets can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points greater in the sample from the subject as compared to the abundance in a control sample. In some aspects, the number of immune cells in the one or more immune cell can be more than 100% in the sample from the subject when compared to the number in a control sample.

In some aspects, a subject can be predicted as having a higher risk for developing and/or diagnosed with irAE associated with ICI treatment, when the immune cell profile comprises one or more immune cell subsets at a greater abundance in the sample from the subject when compared to a control sample, when assessed during ICI treatment. In some aspects, the one or more immune cell subsets when present at higher abundance that is associated with higher risk of developing irAE, when assessed during ICI treatment can comprise one or more of TE CD8 T cells, effector memory (EM) CD8 T cells, CD57lo CD56+TE CD8 T cells, plasmablasts, CLTA-4+naïve B cells, or any combination thereof. In some aspects, the one or more immune cell subsets when present at higher abundance that is associated with higher risk of developing irAE, when assessed during ICI treatment can comprise TE CD8 T cells. In some aspects, the TE CD8 T cells can comprise CD57lo CD56− TE CD8 T cells, CD57hi CD56− TE CD8 T cells, or any combination thereof. In some aspects, the one or more immune cell subsets when present at higher abundance that is associated with higher risk of developing irAE, when assessed during ICI treatment can comprise CD57lo CD56+TE CD8 T cells. In some aspects, the one or more immune cell subsets when present at higher abundance that is associated with higher risk of developing irAE, when assessed during ICI treatment can comprise plasmablasts. In some aspects, one or more immune subsets when present at higher abundance that is associated with higher risk of developing irAE, when assessed during ICI treatment can comprise pDC. In some aspects, the one or more immune cell subsets when present at higher abundance that is associated with higher risk of developing irAE, when assessed during ICI treatment can comprise CLTA-4+ naïve B cells.

In some aspects, the abundance of immune cells in an immune cell subset (e.g., one or more of TE CD8 T cells, effector memory (EM) CD8 T cell, CD57lo CD56+TE CD8 T cells, plasmablasts, CLTA-4+ naïve B cells, or any combination thereof) can be determined from a sample by determining the number of immune cells in the sample that fall into the subset. (For example, X number of the immune cells in the sample are subset 1.) The number of immune cells in a given subset in a sample from the subject can then be compared to the number of immune cells in that subset in a control sample. In some aspects, the abundance (e.g., number) of immune cells in the one or more immune cell subsets associated with risk of irAE, when assessed during ICI treatment can be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in a sample from the subject when compared to abundance in a control sample. In some aspects, the number of immune cells in the one or more immune cell subsets can be at least about 50% greater in a sample from the subject when compared to abundance in a control sample. In some aspects, the number of immune cells in the one or more immune cell subsets can be more than 100% greater in a sample from the subject when compared to the abundance in a control sample.

In some aspects, the abundance of immune cells in an immune cell subset (e.g., one or more of TE CD8 T cells, effector memory (EM) CD8 T cell, CD57lo CD56+TE CD8 T cells, plasmablasts, CLTA-4+ naïve B cells, or any combination thereof) can be determined from a sample by determining the percentage of immune cells in the sample that fall into the subset. (For example, X % of the immune cells in the sample are subset 1.) The percentage of immune cells in a given subset can then be compared to the percentage of immune cells in that subset in a control sample. In some aspects, the abundance (e.g., percentage) of immune cells in the one or more immune cell subsets can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points greater in the sample from the subject as compared to the abundance in a control sample. In some aspects, the number of immune cells in the one or more immune cell subset can be more than 100% in the sample from the subject when compared to the number of immune cells in a control sample.

In another aspect, the present disclosure comprises a method of monitoring the risk of developing irAE associated with ICI treatment in a subject. The method comprises assessing immune cells in a sample provided by the subject and monitoring risk for developing/diagnosing irAE in the subject. The assessment of immune cells comprises determining the abundance of one or more immune cell subsets in the sample. The method further comprises predicting the subject as having a high risk of developing irAE if the abundance of one or more immune cells in Table 1 are different in the sample from the subject when compared to the abundance in a control sample.

In some aspects, a method of monitoring irAE associated with ICI treatment comprise an immune cell profile associated with a baseline or a pre-treatment immune cell profile that correlates with future irAE. In some aspects, the disclosed immune cell profile comprise an interim immune cell profile that correlates with ICI treatment before the onset of irAE. In some aspects, the disclosed immune cell profile comprise one or more immune cell subsets that correlates with onset of irAE during treatment. In some aspects, the disclosed immune cell subsets have elevated abundance which appear at baseline or pre-treatment and sustain during irAE. In some aspects, the disclosed immune cell subsets can have elevated abundance after onset irAE or during later part of the ICI treatment. In some aspects, the elevated abundance of one or more immune cell subsets can reduce in the subject after treatment with irAE mitigating therapy.

In some aspects of the present disclosure, a subject being monitored can have a higher risk for developing irAE associated with immune checkpoint inhibitor (ICI) treatment, when the immune cell profile comprises one or more immune cell subsets at a lower abundance in the sample from the subject when compared to a control sample. In some aspects, the one or more immune cell subsets related to irAE associated with ICI treatment that are present at lower abundance can comprise one or more of naïve B cells, CD57hiCD56+TE CD8 T cells, memory B cells, gamma delta T cells, naïve CD8 T cells, naïve CD4 T cells, or any combination thereof. In some aspects, the one or more immune cell subsets related to irAE associated with ICI treatment that are present at lower abundance can comprise naïve B cells. In some aspects, the one or more immune cell subsets related to irAE associated with ICI treatment that are present at lower abundance can comprise CD57hiCD56+TE CD8 T cells. In some aspects, the one or more immune cell subsets related to irAE associated with ICI treatment that are present at lower abundance can comprise memory B cells. In some aspects, the one or more immune cell subsets related to irAE associated with ICI treatment that are present at lower abundance can comprise gamma delta T cells. In some aspects, the one or more immune cell subsets related to irAE associated with ICI treatment that are present at lower abundance can comprise naïve CD8 T cells. In some aspects, the one or more immune cell subsets related to irAE associated with ICI treatment that are present at lower abundance can comprise naïve CD4 T cells.

In some aspects, during monitoring the abundance of immune cells in an immune cell subset (e.g., one or more of naïve B cells, CD57hiCD56+TE CD8 T cells, memory B cells, gamma delta T cells, naïve CD8 T cells, naïve CD4 T cells, or any combination thereof) can be determined from a sample by determining the number of immune cells in the sample that fall into the subset. (For example, X of the immune cells in the sample are subset 1.) The number of immune cells in a given subset in the sample from the subject can then be compared to the number of immune cells in that subset in a control sample. In some aspects, during assessment, the abundance (e.g., number) of immune cells in the one or more immune cell subset can be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower in the sample from the subject as compared to the abundance of the immune cells in the one or more immune cell subsets in a control sample. In some aspects, number of the immune cells in the one or more immune cell subset can be at least about 25% lower in the sample from the subject as compared to the number in a control sample.

In some aspects, during monitoring, the abundance of immune cells in an immune cell subset (e.g., one or more of naïve B cells, CD57hiCD56+TE CD8 T cells, memory B cells, gamma delta T cells, naïve CD8 T cells, naïve CD4 T cells, or any combination thereof) can be determined from a sample by determining the percentage of immune cells in the sample that fall into the subset. (For example, X % of the immune cells in the sample are subset 1.) The percentage of immune cells in a given subset in a sample from the subject can then be compared to the percentage of immune cells in that subset in a control sample. In some aspects, the abundance (e.g., percentage) of immune cells in the one or more immune cell subset can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points lower in the sample from the subject as compared to the abundance of the immune cells in the one or more immune cell subset in a control sample. In some aspects, number of the immune cells in the one or more immune cell subsets can be at least about 10 percentage points lower in the sample from the subject as compared to the number in a control sample.

In some aspects, a subject being monitored can have a higher risk for developing irAE associated with ICI treatment, when the immune cell profile comprises one or more immune cell subsets at a greater abundance in the sample from the subject when compared to the abundance in a control sample, before the initiation of ICI treatment. In some aspects, the pre-treatment or baseline immune cell subsets when present at higher abundance that is associated with higher risk of developing irAE associated with ICI treatment can comprise one or more of TE CD8 T cells, effector memory (EM) CD8 T cells, CD57lo CD56+TE CD8 T cells, or any combination thereof. In some aspects, the pre-treatment or baseline immune cell subset when present at higher abundance that is associated with higher risk of developing irAE associated with ICI treatment can comprise TE CD8 T cells. In some aspects, TE CD8 T cells can comprise CD57lo CD56− TE CD8 T cells, CD57 h iCD56− TE CD8 T cells, or any combination thereof. In some aspects, the pre-treatment or baseline immune cell subsets when present at higher abundance that is associated with higher risk of developing irAE associated with ICI treatment can comprise effector memory (EM) CD8 T cells. In some aspects, the pre-treatment or baseline immune cell subsets when present at higher abundance that is associated with higher risk of developing irAE associated with ICI treatment can comprise CD57lo CD56+TE CD8 T cells.

In some aspects, during monitoring the abundance of immune cells in an immune cell subset (e.g., one or more of TE CD8 T cells, effector memory (EM) CD8 T cell, CD57lo CD56+TE CD8 T cells, or any combination thereof) can be determined from a sample by determining the number of immune cells in the sample that fall into the subset. (For example, X number of the immune cells in the sample are subset 1.) The number of immune cells in a given subset in a sample from the subject can then be compared to the number of immune cells in that subset in a control sample. In some aspects, the abundance (e.g., number) of immune cells in the one or more immune cell subsets associated with risk of irAE, when assessed during ICI treatment can be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in the sample from the subject as compared to the abundance in a control sample. In some aspects, the number of immune cells in the one or more immune cell subsets can be at least about 50% greater in the sample from the subject compared to the number in a control sample. In some aspects, the number of immune cells in the one or more immune cell subsets can be more than 100% greater in the sample from the subject when compared to a control sample.

In some aspects, during monitoring, the abundance of immune cells in an immune cell subset (e.g., one or more of TE CD8 T cells, effector memory (EM) CD8 T cell, CD57loCD56+TE CD8 T cells, or any combination thereof) can be determined from a sample by determining the percentage of immune cells in the sample that fall into the subset. (For example, X % of the immune cells in the sample are subset 1.) The percentage of immune cells in a given subset in a sample from the subject can then be compared to the percentage of immune cells in that subset in a control sample. In some aspects, the abundance (e.g., percentage) of immune cells in the one or more immune cell subsets can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points greater in the sample from the subject as compared to the abundance in a control sample. In some aspects, the number of immune cells in the one or more immune cell can be more than 100% in the sample from the subject when compared to the number in a control sample.

In some aspects, a subject being monitored can have a higher risk for developing irAE associated with ICI treatment, when the immune cell profile comprises one or more immune cell subsets at a greater abundance in the sample from a subject when compared to the abundance in a control sample, when assessed during ICI treatment. In some aspects, immune cell subsets when present at higher abundance that is associated with higher risk of developing irAE, when assessed during ICI treatment can be one or more of TE CD8 T cells, effector memory (EM) CD8 T cells, CD57lo CD56+TE CD8 T cells, plasmablasts, CLTA-4+ naïve B cells, or any combination thereof. In some aspects, TE CD8 T cells can comprise CD57lo CD56− TE CD8 T cells, CD57hi CD56− TE CD8 T cells, terminal effector CD8 T, pDC, or any combination thereof. In some aspects, the immune cell subset when present at higher abundance that is associated with higher risk of developing irAE associated with ICI treatment, when assessed during ICI treatment, can comprise TE CD8 T cells. In some aspects, TE CD8 T cells can comprise CD57lo CD56− TE CD8 T cells, CD57hi CD56− TE CD8 T cells, or any combination thereof. In some aspects, the immune cell subset when present at higher abundance that is associated with higher risk of developing irAE associated with ICI treatment, when assessed during ICI treatment, can comprise effector memory (EM) CD8 T cells. In some aspects, the immune cell subset when present at higher abundance that is associated with higher risk of developing irAE associated with ICI treatment, when assessed during ICI treatment, can comprise CD57lo CD56+TE CD8 T cells. In some aspects, the immune cell subset when present at higher abundance that is associated with higher risk of developing irAE associated with ICI treatment, when assessed during ICI treatment, can comprise plasmablasts. In some aspects, the immune sell subset when present at higher abundance that is associated with higher risk of developing irAE associated with ICI treatment when assessed during ICI treatment, can comprise pDC. In some aspects, the immune cell subset when present at higher abundance that is associated with higher risk of developing irAE associated with ICI treatment, when assessed during ICI treatment, can comprise CLTA-4+ naïve B cells.

In some aspects, during monitoring, the abundance of immune cells in an immune cell subset (e.g., one or more of TE CD8 T cells, effector memory (EM) CD8 T cell, CD57lo CD56+TE CD8 T cells, plasmablasts, CLTA-4+ naïve B cells, or any combination thereof) can be determined from a sample by determining the number of immune cells in the sample that fall into the subset. (For example, X number of the immune cells in the sample are subset 1.) The number of immune cells in a given subset in a sample from the subject can then be compared to the number of immune cells in that subset in a control sample. In some aspects, the abundance (e.g., number) of immune cells in the one or more immune cell subsets associated with risk of irAE, when assessed during ICI treatment can be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in a sample from the subject as compared to the abundance in a control sample. In some aspects, the number of immune cells in the one or more immune cell subsets can be at least about 50% greater in the sample from the subject when compared to the number in a control sample. In some aspects, the number of immune cells in the one or more immune cell subsets can be more than 100% greater in the sample from the subject when compared to the number in a control sample.

In some aspects, during monitoring, the abundance of immune cells in an immune cell subset (e.g., one or more of TE CD8 T cells, effector memory (EM) CD8 T cell, CD57lo CD56+TE CD8 T cells, plasmablasts, CLTA-4+ naïve B cells, or any combinations there) can be determined from a sample by determining the percentage of immune cells in the sample that fall into the subset. (For example, X % of the immune cells in the sample are subset 1.) The percentage of immune cells in a given subset in the sample from the subject can then be compared to the percentage of immune cells in that subset in a control sample. In some aspects, the abundance (e.g., percentage) of immune cells in the one or more immune cell subsets can be present at least about 0.1 percentage points (for example, 1.0% as compared to 1.1%), at least about 0.2 percentage points, at least about 0.3 percentage points, at least about 0.4 percentage points, at least about 0.5 percentage points, at least about 0.6 percentage points, at least about 0.7 percentage points, at least about 0.8 percentage points, at least about 0.9 percentage points, at least about 1 percentage point, at least about 2 percentage points, at least about 3 percentage points, at least about 4 percentage points, at least about 5 percentage points, at least about 6 percentage points, at least about 7 percentage points, at least about 8 percentage points, at least about 9 percentage points, at least about 10 percentage points, at least about 15 percentage points, at least about 20 percentage points, at least about 25 percentage points, at least about 30 percentage points, at least about 35 percentage points, at least about 40 percentage points, at least about 45 percentage points, at least about 50 percentage points, at least about 55 percentage points, at least about 60 percentage points, at least about 65 percentage points, at least about 70 percentage points, at least about 75 percentage points, at least about 80 percentage points, at least about 85 percentage points, at least about 90 percentage points, at least about 95 percentage points, at least about 99 percentage points, or at least about 100 percentage points greater in the sample from the subject as compared to the abundance in the control sample. In some aspects, the number of immune cells in the one or more immune cell in the sample from the subject can be more than 100% when compared to the number in a control sample.

In some aspects, the disclosed method to assess the risk of a subject developing irAE comprises assessing risk of irAE associated with ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof. In some aspects, ocular toxicity can be one or more of uveitis, dry eye syndrome, ocular myasthenia, myositis, ophthalmoplegia, ptosis, Sjogren's, metastases to the vitreous, vasculitis, damage to rod/cone function, macular edema, retinopathy, choroidal neovascularization, chorioretinal atrophy, vascular leakage, choroidal sarcoid granulomas, choroidal detachment, development of subretinal fluid, optic neuritis, nerve palsies associated with eye, arteritic ischemic optic neuropathy, eye inflammation, or any combination thereof.

In some aspects, the immune cell profile (e.g., abundance of one or more immune cell subsets) can be used for monitoring irAE associated with ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof. In some aspects, ocular toxicity comprise uveitis, dry eye syndrome, ocular myasthenia, myositis, ophthalmoplegia, ptosis, Sjogren's, metastases to the vitreous, vasculitis, damage to rod/cone function, macular edema, retinopathy, choroidal neovascularization, chorioretinal atrophy, vascular leakage, choroidal sarcoid granulomas, choroidal detachment, development of subretinal fluid, optic neuritis, nerve palsies associated with eye, arteritic ischemic optic neuropathy, eye inflammation, or any combination thereof.

Immune cell subsets can be determined, detected, quantified, and/or monitored using well known methods in the art including immune profiling assay, mass cytometry (cytometry by time-of-flight CyTOF), flow cytometry and cell sorting including FACS and immunomagnetic separation. In some aspects, immune cell subsets are determined/detected using high-dimensional mass cytometry (cytometry by time-of-flight (CyTOF)). In some aspects, the abundance of the one or more immune cell subsets can be determined as the abundance of an immune cell subset determined as percentage of total PBMC cells, or as the abundance of cell subsets as a percentage of total CD45+ cells. Samples to be tested comprise whole blood, serum, plasma, urine, CSF or other suitable body fluid. In some aspects, the sample can be a peripheral blood sample. In some aspects, the sample can be peripheral blood mononuclear cells (PBMCs). Samples can be obtained from the subject before, during and/or after ICI treatment, and immune cell subsets are identified to assess the risk of irAE. In some aspects, samples can be obtained before the initiation of ICI treatment, two weeks, six weeks, three months, every three months thereafter, and after initiation of steroids for irAE treatment. The risk assessment may comprise predicting, diagnosing, or monitoring irAE.

In some aspects, the disclosed method for predicting, diagnosing or monitoring irAE using immune cell profile can comprise repeating one or more steps of a disclosed method and/or modifying one or more steps of a disclosed method (such as, for example, treatment step). In an aspect, the disclosed method can further comprise of treating cancer and/or modifying one or more of the administrations of the ICI compounds. In some aspects the method, can comprise modifying one or more of steps of administration. For example, in an aspect, the method can be altered by changing the amount of one or more of the ICI compounds administered to a subject, or by changing the frequency of administration of one or more of the ICI compounds to a subject, or by changing the duration of time one or more of the ICI compounds administered to a subject.

In some aspects, assessment of one or more of the disclosed immune cell subsets can be combined with assessment of one or more autoantibodies and/or one or more cytokines for predicting, diagnosing, or monitoring irAE associated with irAE, before, during or after ICI treatment. The combination of identification of one or more of the immune cell subsets with identification of one or more autoantibodies and/or one or more cytokines can be used before administering ICI treatment as a baseline or a pre-treatment profile that correlates with future irAE. Additionally, the combination of identification one or more of the immune cell subsets with identification one or more of autoantibodies and/or cytokines can be used during ICI treatment to predict or diagnose irAE during treatment. In some aspects, the combination of the identification of one or more of the immune cell subsets with identification of one or more of autoantibodies and/or cytokines can be used after ICI treatment to assess irAE after treatment.

### Autoantibody Profiles

In some aspects, the present disclosure provides an autoantibody profile associated with ICI-related irAE. In some aspects, the autoantibody profile comprises one or more autoantibodies with an elevated level in a sample from the subject with irAE when compared to the level in a control sample.

In some aspects, the autoantibodies may comprise one or more of the autoantibodies disclosed in U.S. patent application Ser. No. 16/487,335 (U.S. Patent Application Publication No. US 2020/0284803), the disclosures of which are incorporated by reference in its entirety.

In some aspects, the autoantibodies can comprise angiotensin receptor (AGTR), α-actin, U1-snRNP A, amyloid, collagen II, or any combination thereof.

In some aspects, the present disclosure provides a method of predicting the risk of developing and/or diagnosing irAE associated with ICI treatment in a subject. The method comprises providing a sample from the subject, assessing the level of one or more autoantibodies in the sample, and predicting risk for developing/diagnosing irAE in the subject.

In some aspects, the method further comprises predicting the subject as having a high risk of developing or diagnosed with having irAE if the level of one or more autoantibodies are different in the sample from the subject when compared to the level in a control sample as described in U.S. patent application Ser. No. 16/487,335 (U.S. Patent Application Publication No. US 2020/0284803), the disclosures of which are incorporated by reference in its entirety.

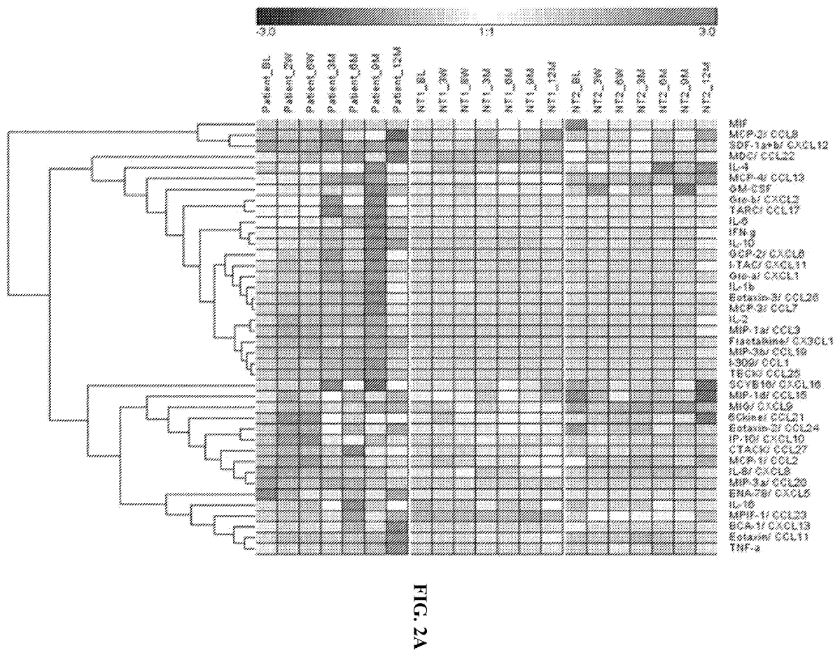

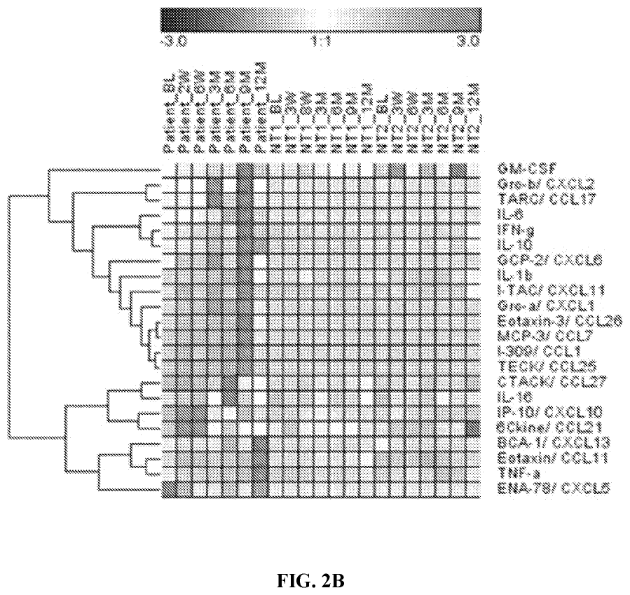

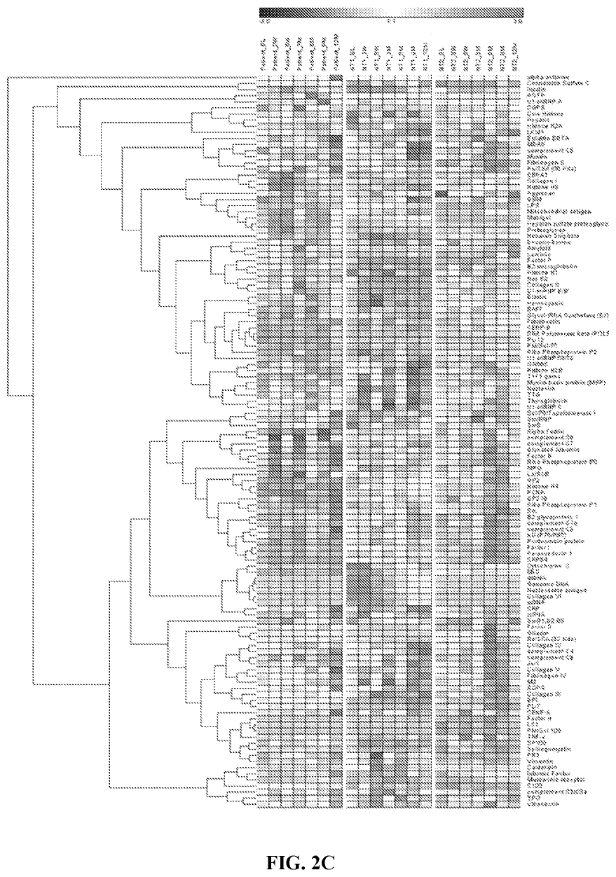

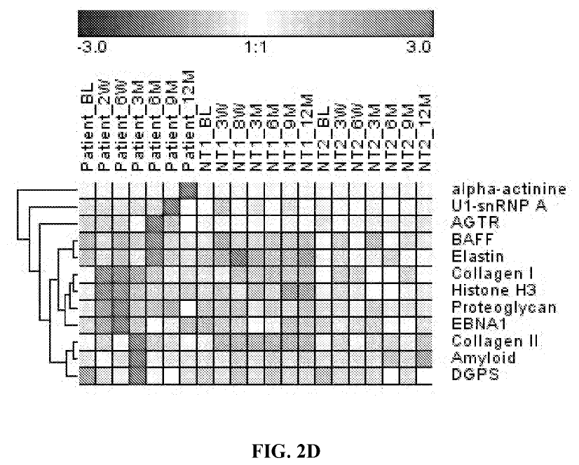

In some aspects, the method further comprises predicting the subject as having a high risk of developing or diagnosed with having irAE if the level of one or more autoantibodies in FIG. 2D are different in the sample from the subject when compared to the level in a control sample.

In some aspects, method of predicting the risk of developing and/or diagnosing irAE associated with ICI treatment comprise assessing the level of angiotensin receptor (AGTR), α-actin, U1-snRNP A, amyloid, collagen II, B-cell activating factor (BAFF), elastin, collagen I, histone H3, proteoglycan, Epstein-Barr nuclear antigen 1 (EBNA1), DGPS, or any combination thereof. In some aspects, the autoantibody can comprise angiotensin receptor (AGTR), α-actin, U1-snRNP A, amyloid, collagen II or any combination thereof. In some aspects, the autoantibody can comprise angiotensin receptor (AGTR). In some aspects, the autoantibody can comprise α-actin. In some aspects, the autoantibody can comprise U1-snRNP A. In some aspects, the autoantibody can comprise amyloid. In some aspects, the autoantibody can comprise collagen II. In some aspect, the autoantibody can comprise BAFF. In some aspect, the autoantibody can comprise elastin. In some aspect, the autoantibody can comprise collagen I. In some aspect, the autoantibody can comprise Histone H3. In some aspect, the autoantibody can comprise proteoglycan. In some aspect, the autoantibody can comprise EBNA1. In some aspect, the autoantibody can comprise DGPS.

In some aspects, method of predicting the risk of developing and/or diagnosing irAE associated with ICI treatment comprise an autoantibody profile associated with a baseline or a pre-treatment autoantibody profile that correlates with future irAE. In some aspects, the disclosed autoantibody profile comprise an interim autoantibody profile that correlates with ICI treatment before the onset of irAE. In some aspects, the disclosed autoantibody profile comprise one or more autoantibodies that correlates with onset of irAE during treatment. In some aspects, the disclosed autoantibodies have elevated expression which appear at baseline or pre-treatment and sustain during irAE. In some aspects, the disclosed autoantibodies have elevated level after onset irAE or during later part of the ICI treatment. In some aspects, the elevated level of one or more autoantibodies can reduce in the subject after treatment with irAE mitigating therapy. In some aspects, the panel of autoantibodies comprises AGTR, α-actin, U1-snRNP A, amyloid, collagen II, BAFF, elastin, collagen I, histone H3, proteoglycan, EBNA1, DGPS, or any combination thereof.

In some aspects, during predicting or diagnosing the risk of irAE, one or more of the disclosed autoantibodies have an elevated level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in the sample from the subject when compared to the level in a control sample. In some aspects, the disclosed autoantibodies can have an elevated level greater than 100% in a sample from the subject when as compared to the level in a control sample.

In some aspects, during assessment, one or more autoantibodies can have a level at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower in the sample from the subject when compared to the level in a control sample. In some aspects, one or more autoantibodies can have a level at least about 25% lower, at least 50% lower, or 100% lower in the sample from the subject, when compared to the level in a control sample.

In some aspects, the disclosure provides a method of monitoring the risk of developing irAE associated with ICI treatment in a subject. The method comprises providing a sample from the subject, assessing the level of one or more autoantibodies in the sample, and monitoring risk for developing/diagnosing irAE in the subject. The method further comprises predicting the subject as having a high risk of developing irAE if the level of one or more autoantibodies in FIG. 2D are different in the sample from the subject when compared to the level in a control sample.

In some aspects, method of monitoring irAE associated with ICI treatment comprises assessing the level of one or more autoantibodies comprising AGTR, α-actin, U1-snRNP A, amyloid, collagen II, BAFF, elastin, collagen I, Histone H3, proteoglycan, EBNA1, DGPS, or any combination thereof. In some aspects, the autoantibody comprises angiotensin receptor (AGTR), α-actin, U1-snRNP A, amyloid, collagen II or any combination thereof. In some aspects, the autoantibody comprises AGTR. In some aspects, the autoantibody comprises α-actin. In some aspects, the autoantibody comprises U1-snRNP A. In some aspects, the autoantibody comprises amyloid. In some aspects, the autoantibody comprises collagen II. In some aspect, the autoantibody comprises BAFF. In some aspect, the autoantibody comprises elastin. In some aspect, the autoantibody comprises collagen I. In some aspect, the autoantibody comprises histone H3. In some aspect, the autoantibody comprises proteoglycan. In some aspect, the autoantibody comprises EBNA1. In some aspect, the autoantibody comprises DGPS.

In some aspects, method of monitoring irAE associated with ICI treatment comprise an autoantibody profile associated with a baseline or a pre-treatment autoantibody profile that correlates with future irAE. In some aspects, the disclosed autoantibody profile comprise an interim autoantibody profile that correlates with ICI treatment before the onset of irAE. In some aspects, the disclosed autoantibody profile comprise one or more autoantibodies that correlate with onset of irAE during treatment. In some aspects, the disclosed autoantibodies can have elevated level which appear at baseline or pre-treatment and sustain during irAE. In some aspects, the disclosed autoantibodies can have elevated level after onset irAE or during later part of the ICI treatment. In some aspects, the elevated level of one or more autoantibodies can reduce in the subject after treatment with irAE mitigating therapy. In some aspects, the panel of autoantibody comprises AGTR, α-actin, U1-snRNP A, amyloid, collagen II, BAFF, elastin, collagen I, histone H3, proteoglycan, EBNA1, DGPS, or any combination thereof.

In some aspects, during monitoring the risk of irAE, one or more of the disclosed one or more autoantibodies have an elevated level at at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in a sample from the subject when compared to the level in a control sample. In some aspects, the disclosed one or more autoantibodies can have an elevated level at greater than 100% in the sample from the subject compared to the level in a control sample.

In some aspects, during monitoring, one or more autoantibodies can be present at a level of at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% lower in the sample from the subject as compared to the level in a control sample. In some aspects, one or more autoantibodies can have a level at least about 25% lower, at least 50% lower, or more than 100% lower, in a sample from the subject when compared to the level in a control sample.

Isolating, purifying, measuring and/or monitoring the level of one or more autoantibodies can be performed using any known method in the art. By way of non-limiting examples, one or more autoantibodies can be detected using enzyme linked immunosorbent assay (ELISA), radioimmunoassay (RIA), immunoradiometric assay, fluoroimmunoassay, chemiluminescent assay, bioluminescent assay, and Western blot. In some aspects, autoantibody profiling can be done using protein array panel comprising various antigens. By the way of non-limiting example, protein array panel can be a custom protein array panel of autoantigens, including nuclear antigens, cytosolic/matrix antigens, and tissue/organ-specific antigens. Samples to be tested comprise of whole blood, serum, plasma, urine, CSF or other suitable body fluid. Samples can be obtained from the subject before, during and/or after ICI treatment, and quantification of the level of one or more autoantibodies can be performed, to assess the risk of irAE. In some aspects, one or more autoantibodies can be measured in a blood plasma sample. In some aspects, the sample can be a peripheral blood sample. The risk assessment comprises predicting, diagnosing, or monitoring ICI-related ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic iymphohistiocytosis/macrophage activation syndrome (HLH/MAS), aplastic anemia, pure red cell aplasia, neutropenia, or any combination thereof.

### Cytokine Profile

In some aspects, the present disclosure provides cytokine profile associated with ICI-related irAE. In some aspects, the cytokine profile comprises one or more cytokines with an elevated expression in a subject with irAE when compared to the sample from the subject when compared to the expression in a control sample. In some aspects, cytokine profile comprises one or more cytokines with lower expression in the sample from the subject with irAE, when compared to the expression in a control sample. In some aspects, the cytokine profile comprise a baseline or a pre-treatment cytokine profile that correlates with future irAE. In some aspects, the cytokine profile comprise an interim cytokine profile that correlates with ICI treatment before the onset of irAE. In some aspects, the cytokine profile comprises one or more cytokines that correlates with onset of irAE during ICI treatment. In some aspects, the disclosed cytokines are used as biomarkers to assess the risk of a subject developing irAE before, during or after ICI treatment.

In some aspects, the disclosed cytokines may comprise one or more of the cytokines disclosed in U.S. patent application Ser. No. 14/045,482 (U.S. Patent Application Publication No. US 2021/0263045), the disclosures of which are incorporated by reference in its entirety.

In some aspects, the expression of one or more cytokines can be determined as expression level of the gene encoding the cytokine. In some aspects, the expression of one or more cytokines can be determined as the concentration of the corresponding protein of the cytokine.

In some aspects, the disclosed cytokines are one or more of the cytokines disclosed in FIG. 2A or FIG. 2B. In some aspects, the disclosed cytokine is MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-a, or any combination thereof. In some aspects, the cytokines are one or more of GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof. In some aspects, the cytokine comprise at least IL-6, IL-1β, TNFα, IFNγ, CXCL2, CCL17, or any combination thereof. In some aspects, the cytokine comprise IL-6. In some aspects, the cytokine comprise IL-1β. In some aspects, the cytokine comprise TNFα. In some aspects, the cytokine comprise IFNγ. In some aspects, the cytokine comprise CXCL2. In some aspects, the cytokine comprise CCL17.

In some aspects, the present disclosure provides a method of predicting the risk of developing and/or diagnosing irAE associated with ICI treatment in a subject. The method comprises providing a sample from the subject, assessing the expression of one or more cytokines in the sample from the subject, and predicting risk for developing/diagnosing irAE in the subject.

In some aspects, the method further comprises predicting the subject as having a high risk of developing or diagnosed with having irAE if the expression of one or more cytokines are different in the sample from the subject when compared to the expression in a control sample as described in U.S. patent application Ser. No. 14/045,482 (U.S. Patent Application Publication No. US 2021/0263045), the disclosures of which are incorporated by reference in its entirety.

In some aspects, the method further comprises predicting the subject as having a high risk of developing or diagnosed with having irAE if the expression of one or more cytokines in FIG. 2A or FIG. 2B are different in the sample from the subject when compared to the expression in a control sample. In some aspects, the method comprises predicting the subject as having a high risk of developing or diagnosed with having irAE if the expression of IL-6, IL-1β, TNFα, IFNγ, CXCL2, CCL17, or any combination thereof, are different in the sample from the subject when compared to the expression in a control sample.

In some aspects, method of predicting the risk of developing and/or diagnosing irAE associated with ICI treatment comprise assessing the expression of one or more of MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-a, or any combination thereof. In some aspects, the disclosed cytokine comprise GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof.

In some aspects, method of predicting the risk of developing and/or diagnosing irAE associated with ICI treatment comprise a cytokine profile associated with a baseline or a pre-treatment cytokine profile that correlates with future irAE. In some aspects, the disclosed cytokine profile comprise an interim cytokine profile that correlates with ICI treatment before the onset of irAE. In some aspects, the disclosed cytokine profile comprise one or more cytokines that correlates with onset of irAE during treatment. In some aspects, the disclosed one or more cytokines can have elevated expression which appear at baseline or pre-treatment and sustain during irAE. In some aspects, the disclosed one or more cytokines can have elevated expression after onset irAE or during later part of the ICI treatment. In some aspects, the elevated expression of one or more cytokines can reduce in the subject after treatment with irAE mitigating therapy.

In some aspects, the one or more of the disclosed one or more cytokines have an elevated expression of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in the sample from the subject when compared to the expression in a control sample.

In some aspects, during predicting or diagnosing the risk of irAE predicting or diagnosing the risk of irAE one or more of the disclosed cytokines have an elevated expression having a log fold change value from about 0.1 to about 5. For example, a log fold change value can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.

In some aspects, during predicting or diagnosing the expression of one or more of the disclosed cytokines can be measured by determining the concentration of the cytokine. In such aspects, the concentration of the cytokines can be determined using a standard curve. In some aspects, the concentration of the cytokines can be determined using a fit of a standard curve such as for e.g., mean fluorescence intensity versus pg/ml. In some aspects, during prediction or diagnosis, one or more of the disclosed cytokines have a concentration of about 10 pg/ml, about 20 pg/ml, about 30 pg/ml, about 40 pg/ml, about 50 pg/ml, about 60 pg/ml, about 70 pg/ml, about 80 pg/ml, about 90 pg/ml, about 100 pg/ml, about 120 pg/ml, about 140 pg/ml, about 150 pg/ml, about 160 pg/ml, about 180 pg/ml, about 200 pg/ml, about 300 pg/ml, about 400 pg/ml, about 500 pg/ml, about 600 pg/ml, about 700 pg/ml, or about 800 pg/ml.

In some aspects, the disclosure provides a method of monitoring the risk of developing irAE associated with ICI treatment in a subject. The method comprises providing a sample from the subject, assessing expression of one or more cytokines in the sample, and monitoring risk for developing/diagnosing irAE in the subject. The method further comprises predicting the subject as having a high risk of developing rAE if the expression of one or more cytokines in FIG. 2A or FIG. 2B is different in the subject when compared to the expression in a control sample.

In some aspects, method of monitoring irAE associated with ICI treatment comprise assessing the expression of one or more of cytokines MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-a, or any combination thereof. In some aspects, the disclosed cytokine comprise GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof. In some aspects, the disclosed cytokine is IL-6, IL-1β, TNFα, IFNγ, CXCL2, CCL17, or any combination thereof. In some aspects, the cytokine comprises at least IL-6, IL-1β, TNFα, IFNγ, CXCL2, CCL17, or any combination thereof. In some aspects, the cytokine comprises IL-6. In some aspects, the cytokine comprises IL-1β. In some aspects, the cytokine comprise TNFα. In some aspects, the cytokine comprises IFNγ. In some aspects, the cytokine comprises CXCL2. In some aspects, the cytokine comprises CCL17.

In some aspects, the method of monitoring irAE associated with ICI treatment comprise a cytokine profile associated with a baseline or a pre-treatment cytokine profile that correlates with future irAE. In some aspects, the disclosed cytokine profile comprise an interim cytokine profile that correlates with ICI treatment before the onset of irAE. In some aspects, the disclosed cytokine profile comprise one or more cytokines that correlates with onset of irAE during treatment. In some aspects, the disclosed one or more cytokines have an elevated expression which appear at baseline or pre-treatment and sustain during irAE. In some aspects, the disclosed one or more cytokines have an elevated expression after onset irAE or during later part of the ICI treatment. In some aspects, the elevated expression of one or more cytokines can reduce in the subject after treatment with irAE mitigating therapy. In some aspects, the profile of cytokine comprises IL-6, IL-1β, TNFα, IFNγ, CXCL2, CCL17, or any combination thereof.

In some aspects, during monitoring, the expression of one or more cytokines can be determined as expression level of the gene encoding the cytokine. In some aspects, during monitoring, the expression of one or more cytokines can be determined as the concentration of the corresponding protein of the cytokine.

In some aspects, during monitoring the risk of irAE, one or more of the disclosed cytokines have an elevated expression of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater in the sample from the subject when compared to the expression in a control sample. In some aspects, one or more of the disclosed cytokines have an elevated expression greater than 100% in a sample from the subject when compared to the expression in a control sample.

In some aspects, during monitoring the risk of irAE predicting or diagnosing the risk of irAE one or more of the disclosed cytokines have an elevated expression having a log fold change value from about 0.1 to about 5. For example, a log fold change value can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.

In some aspects, during monitoring the expression of one or more of the disclosed cytokines can be measured by determining the concentration of the cytokine. In such aspects, the concentration of the cytokines can be determined using a standard curve. In some aspects, the concentration of the cytokines can be determined using a fit of a standard curve such as for e.g., mean fluorescence intensity versus pg/ml. In some aspects, during prediction or diagnosis, one or more of the disclosed cytokines have a concentration of about 10 pg/ml, about 20 pg/ml, about 30 pg/ml, about 40 pg/ml, about 50 pg/ml, about 60 pg/ml, about 70 pg/ml, about 80 pg/ml, about 90 pg/ml, about 100 pg/ml, about 120 pg/ml, about 140 pg/ml, about 150 pg/ml, about 160 pg/ml, about 180 pg/ml, about 200 pg/ml, about 300 pg/ml, about 400 pg/ml, about 500 pg/ml, about 600 pg/ml, about 700 pg/ml, or about 800 pg/ml.

Quantification and/or monitoring the expression of one or more cytokines can be conducted using well known methods in the art including enzyme linked immunosorbent assay (ELISA), radioimmunoassay (RIA), immunoradiometric assay, fluoroimmunoassay, chemiluminescent assay, bioluminescent assay, and Western blot. In some aspects, monitoring expression of cytokine is performed using readily available cytokine panels (e.g. Bio-Plex Pro Human Chemokine 40-plex Panel, Bio-Rad Laboratories, Hercules, California). Concentrations of one or more cytokines can be determined on the basis of the fit of a provided standard curve for mean fluorescence intensity. Samples to be tested comprise whole blood, serum, plasma, urine, CSF or other suitable body fluid. In some aspects, the sample can be a peripheral blood sample. Samples can be obtained from the subject before, during and/or after ICI treatment, and quantification of concentration of one or more cytokines can be performed, to assess the risk of irAE. The risk assessment comprises predicting, diagnosing, or monitoring ICI-related ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof.

### Combination Profiles

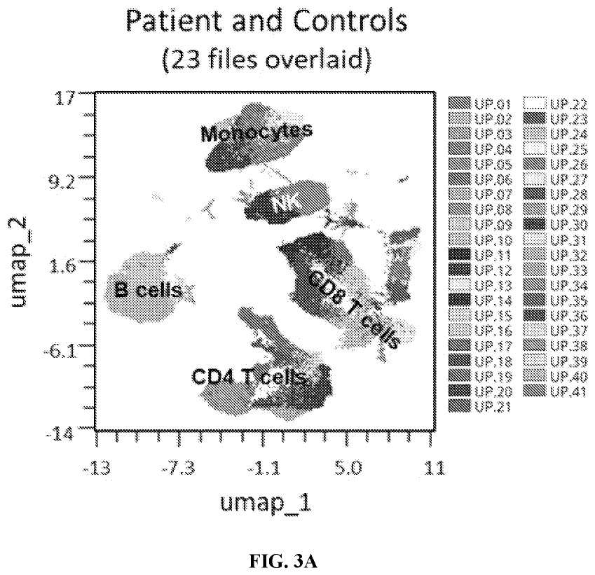

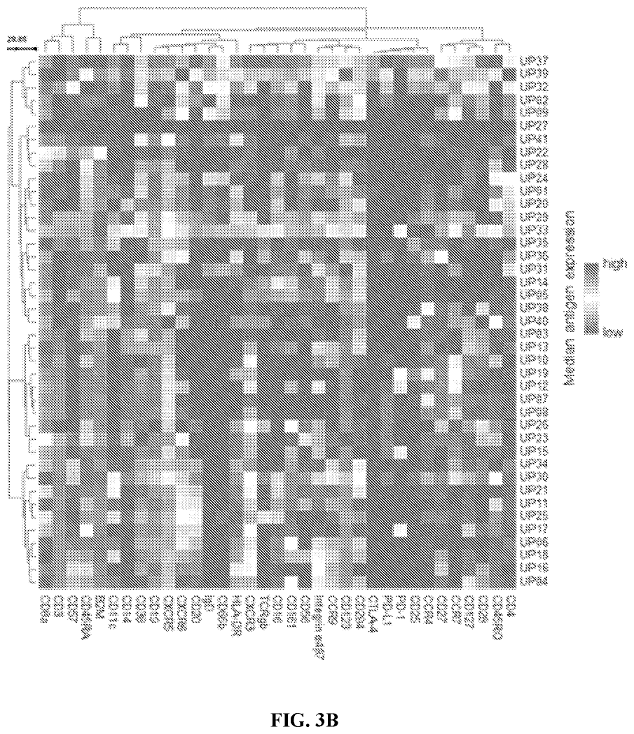

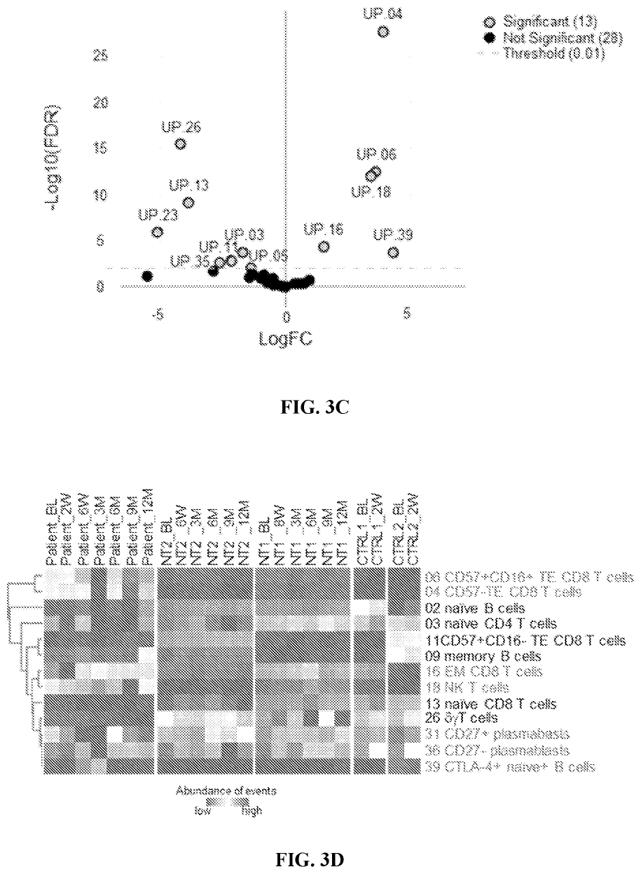

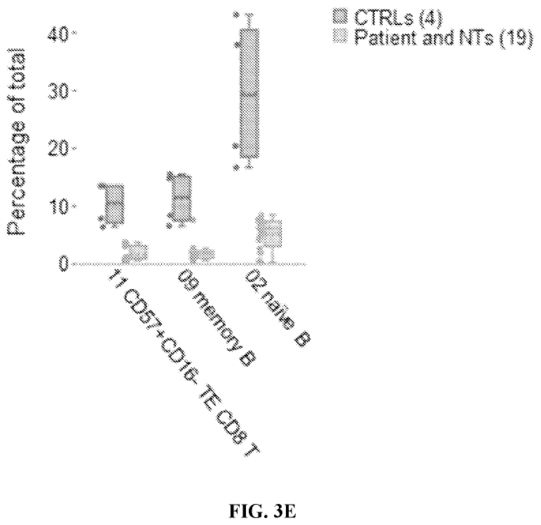

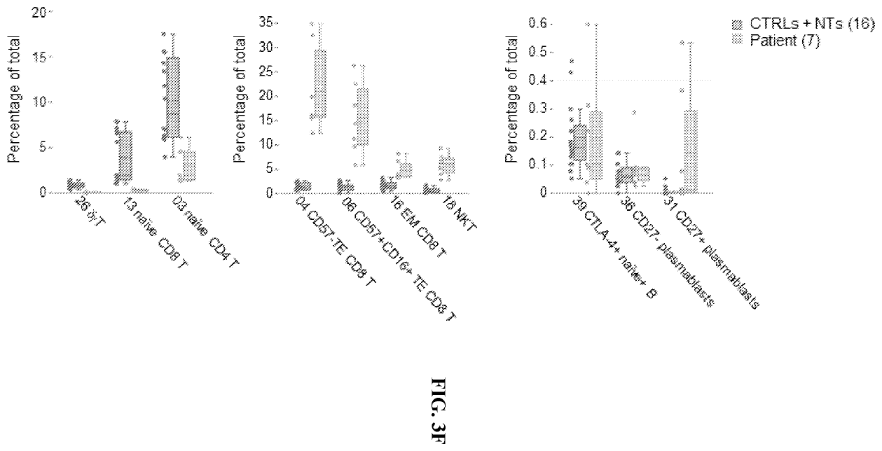

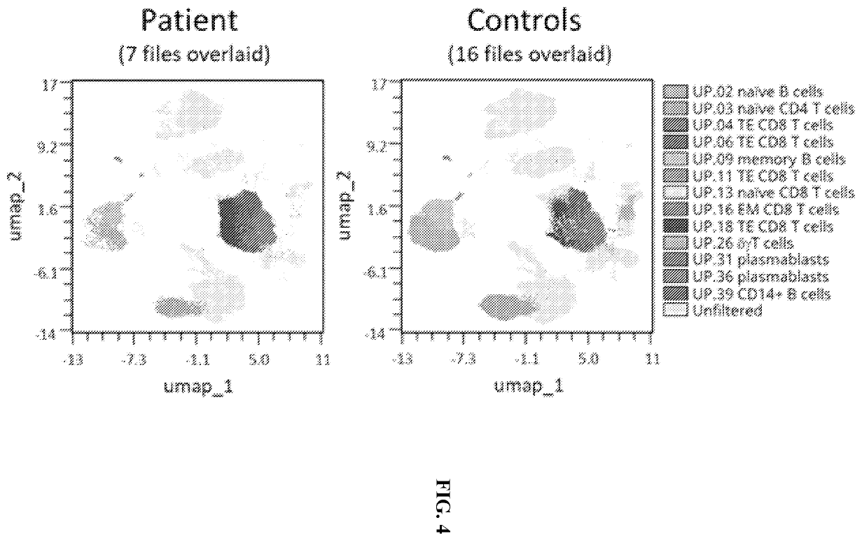

In some aspects, the disclosure further provides a profile which comprises one or more of immune profiles, autoantibody profiles, cytokine profiles, or any combination thereof. The profile comprises one or more of the immune cell subsets, autoantibodies, and/or cytokines provided in Table 1, FIG. 3F, Table 4, FIG. 2D, FIG. 2A, FIG. 2B, U.S. patent application Ser. No. 16/487,335 (U.S. Patent Application Publication No. US 2020/0284803), U.S. patent application Ser. No. 14/045,482 (U.S. Patent Application Publication No. US 2021/0263045), or any combination thereof. In some aspects, the profile comprises a baseline or a pre-treatment immune cell subset abundance, an autoantibody level, a cytokine expression, or any combination thereof, that correlates with future irAE. In some aspects, the profile comprise an interim immune cell subset abundance, an autoantibody level, a cytokine expression, or any combination thereof that correlates with ICI treatment before the onset of irAE. In some aspects, the profile comprises an immune cell subset abundance, an autoantibody level, a cytokine expression, or any combination thereof that correlates with onset of irAE during treatment. In some aspects, the irAE is ocular toxicity.

In some aspects, one or more immune cell subsets, autoantibodies, cytokines, or any combination thereof are used as biomarkers to assess the risk of a subject developing irAE before, during, or after ICI treatment. The method comprises providing a sample from the subject, assessing one or more immune cell subset abundance, autoantibody level, cytokine expression, or any combination thereof, in the sample, and predicting risk for developing/diagnosing irAE in the subject. In some aspects, a subject can be predicted as having a higher risk for developing and/or diagnosed with irAE associated with ICI treatment, when the profile comprises one or more immune cells, autoantibodies, cytokines, or any combination thereof, at a greater abundance, level or expression in a sample from the subject when compared to the abundance, level or expression in a control sample.

In some aspects, higher risk of developing irAE is associated with higher abundance, level, or expression one or more immune cells, autoantibodies, cytokines comprising CD57lo CD56− TE CD8 T cells, CD57hi CD56− TE CD8 T cells, effector memory (EM) CD8 T cell, CD57loCD56+TE CD8 T cells, plasmablasts, pDC, CLTA-4+ naïve B cells, AGTR, α-actin, U1-snRNP A, amyloid, collagen II, BAFF, elastin, collagen I, histone H3, Proteoglycan, EBNA1, DGPS, MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-a, or any combination thereof and/or lower abundance of naïve B cells, CD57hiCD56+TE CD8 T cells, memory B cells, gamma delta T cells, naïve CD8 T cells, naïve CD4 T cells, or any combination thereof. In some aspects, the disclosed profile comprises CD57lo CD56− TE CD8 T cells, CD57hiCD56− TE CD8 T cells, effector memory (EM) CD8 T cell, CD57lo CD56+TE CD8 T cells, plasmablasts, pDC, CLTA-4+ naïve B cells, AGTR, α-actin, U1-snRNP A, amyloid, collagen II, GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof, at elevated abundance, level, or expression and/or gamma delta T cells, naïve CD8 T cells and/or naïve CD4 T cells at lower abundance. In some aspects, the disclosed profile comprise CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, angiotensin receptor (AGTR), α-actin, U1-snRNPA, amyloid, collagen II, IL-6, IL-1β, TNFα, IFNg, CXCL2, CCL17, or any combination thereof, at elevated abundance, level, or expression and/or naïve B cells, CD57hiCD56+TE CD8 T cells, memory B cells, gamma delta T cells, naïve CD8 T cells, naïve CD4 T cells, or any combination thereof at lower abundance.

In some aspects, profile comprising one or more immune cell subsets, autoantibodies, cytokines or any combination thereof can be used for predicting and/or diagnosing irAE associated with ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof. In some aspects, ocular toxicity comprise uveitis, dry eye syndrome, ocular myasthenia, myositis, ophthalmoplegia, ptosis, Sjogren's, metastases to the vitreous, vasculitis, damage to rod/cone function, macular edema, retinopathy, choroidal neovascularization, chorioretinal atrophy, vascular leakage, choroidal sarcoid granulomas, choroidal detachment, development of subretinal fluid, optic neuritis, nerve palsies associated with eye, arteritic ischemic optic neuropathy, eye inflammation, or any combination thereof.

In some aspects, the disclosure provides a method of monitoring the risk of developing irAE associated with ICI treatment in a subject. The method comprises providing a sample from the subject, assessing the abundance, level, or expression of one or more immune cell subsets, autoantibodies, cytokines, or any combination thereof, in the sample, and monitoring risk for developing/diagnosing irAE in the subject. The method further comprises predicting the subject as having a high risk of developing irAE if one or more immune cell subsets, autoantibodies, cytokines, or any combination thereof have elevated abundance, level, or expression in the sample from the subject when compared to the abundance, level, or expression in a control sample. In some aspects, during monitoring, higher risk of developing irAE can be associated with higher abundance, level, or expression of one or more immune cell subsets, autoantibodies, cytokines, comprising one or more of CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, AGTR, α-actin, U1-snRNP A, amyloid, collagen II, BAFF, elastin, collagen I, histone H3, Proteoglycan, EBNA1, DGPS, MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-a, or any combination thereof, and/or lower abundance of naïve B cells, CD57hiCD56+TE CD8 T cells, memory B cells, gamma delta T cells, naïve CD8 T cells, naïve CD4 T cells, or any combination thereof. In some aspects, the disclosed profile comprises CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, AGTR, α-actin, U1-snRNP A, amyloid, collagen II, GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof, at elevated abundance, level, or expression, and/or naïve B cells, CD57hiCD56+TE CD8 T cells, memory B cells, gamma delta T cells, naïve CD8 T cells, naïve CD4 T cells, or any combination thereof, at lower abundance. In some aspects, the disclosed profile comprise CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, angiotensin receptor (AGTR), α-actin, U1-snRNP A, amyloid, collagen II, IL-6, IL-1β, TNFα, IFNg, CXCL2, CCL17, or any combination thereof, at elevated abundance, level, or expression, and/or naïve B cells, CD57hiCD56+TE CD8 T cells, memory B cells, gamma delta T cells, naïve CD8 T cells, naïve CD4 T cells, or any combination thereof, at lower abundance.

In some aspects, the disclosed method for predicting, diagnosing or monitoring irAE using profile comprising one or more immune cell subsets, autoantibodies, cytokines, or any combination thereof can further comprise repeating one or more steps of a disclosed method and/or modifying one or more steps of a disclosed method (such as, for example, treatment step). In an aspect, the disclosed method can further comprise of treating cancer and can comprise modifying one or more of the administrations of the ICI compounds. In some aspects the method, can comprise modifying one or more of steps of administration. For example, in an aspect, the method can be altered by changing the amount of one or more of the ICI compounds, thereof administered to a subject, or by changing the frequency of administration of one or more of the ICI compounds thereof to a subject, or by changing the duration of time one or more of the ICI compounds administered to a subject. In some aspects, the ICI compounds comprise one or more ICI compounds disclosed herein

### III. Methods of Treatment

The present disclosure further provides a method of treating a subject with cancer. The method comprises assessing the abundance of one or more of the immune cell subsets, and assigning a risk level for developing irAE. In some aspects, risk assessment can be done before, during and/or after ICI treatment. In some aspects, the risk assessment comprises predicting, diagnosing, or monitoring ICI-related irAE. If the profile correlates with irAE or future development of irAE, the subjects are assigned a high risk for developing irAE. If the profile correlates with no irAE the subjects are assigned as low risk for developing irAE.

In some aspects, the ICI treatment comprise one or more ICI treatment disclosed herein. The progression of treatment of cancer using ICI can be guided by the profile assessments and assigned risk levels. Based on the profile assessments and assigned risk levels, the ICI treatment can be continued, withdrawn, or modified accordingly.

In some aspects, the method for treating cancer comprises providing a sample from a subject, assessing one or more of the immune cell subsets, autoantibodies, or cytokines or any combination thereof, treating the subject with an ICI treatment if subject is predicted, diagnosed, or monitored as low risk for irAE. In some aspects, the subject receives a non-ICI treatment if the subject is predicted, diagnosed or monitored as high risk. In some aspects, the subject receives an ICI treatment and an irAE mitigating treatment if the subject is diagnosed as high risk.

In some aspects, ICI treatment comprises administration of an inhibitor of PD-1, PD-L1, TIM-3, LAG-3, CTLA-4, CSF-1R, or any combination thereof. In some aspects, exemplary irAE mitigating therapy can include corticosteroids (e.g., prednisone, methylprednisolone, dexamethasone, budesonide), TNF inhibitors (e.g., infliximab), or hormone replacement (e.g., hydrocortisone, levothyroxine), and CXCL8 inhibitors (e.g. repertaxin).

In some aspects, the methods disclosed herein to assess the risk of a subject developing irAE can comprise assessing the risk of one or more other ICI associated irAE including ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, diarrhea or another gastrointestinal disorder, pure red cell aplasia, microcytic anemia, lupus, autoimmune nephritism, autoimmune hepatitis, pneumonitis, pericarditis, endocrinopathy, Addison's disease, hypogonadism, Sjogren's syndrome, and/or type I diabetes.

In some aspects, based on the severity of irAE, ICI treatment may be suspended, with consideration of resuming when symptoms of irAE revert. In some aspects, ICI treatment administered to a subject may be modified. In such aspects, the dosage of ICI treatment may be reduced or skipped. Additionally, irAE mitigating therapy for e.g., corticosteroids may be administered. Subjects may be administered a high-dose corticosteroids (for e.g., prednisone 1 to 2 mg/kg/d or methylprednisolone 1 to 2 mg/kg/d), which may be tapered over the course of at least 4 to 6 weeks. In some aspects, infliximab or other immunosuppressive therapy may be administered either individually, or in combination with other irAE mitigating therapies. In some aspects, irAE mitigating therapy may be administered sequentially or simultaneously with the ICI treatment. In some aspects, permanent discontinuation of ICI may be recommended. In some aspects, a non-ICI treatment may be recommended.

In some aspects, corticosteroids include, for example, betamethasone sodium phosphate, desonide sodium phosphate, dexamethasone sodium phosphate, hydrocortisone sodium phosphate, hydrocortisone sodium succinate, methylprednisolone disodium phosphate, methylprednisolone sodium succinate, prednisolone sodium phosphate, prednisolone sodium succinate, prednisolamate hydrochloride, prednisone disodium phosphate, prednisone sodium succinate, triamcinolone acetonide disodium phosphate and triamcinolone acetonide disodium phosphate, alclomethasone dipropionate, amcinonide, beclomethasone monopropionate, betamethasone 17-valerate, ciclomethasone, clobetasol propionate, clobetasone butyrate, deprodone propionate, desonide, desoxymethasone, dexamethasone acetate, diflucortolone valerate, diflurasone diacetate, diflucortolone, difluprednate, flumetasone pivalate, flunisolide, fluocinolone acetonide acetate, fluocinonide, fluocortolone pivalate, fluormetholone acetate, fluprednidene acetate, halcinonide, halometasone, hydrocortisone acetate, medrysone, methylprednisolone acetate, mometasone furoate, parametasone acetate, prednicarbate, prednisolone acetate, prednylidene, rimexolone, tixocortol pivalate, and triamcinolone hexacetonide.

In some aspects, an irAE mitigating treatment can comprise use of cytokine inhibitors that inhibit one or more cytokines or their respective cytokine receptors. For example, the receptor CXCR2 has the cytokine ligands of CXCL1, CXCL2, CXCL3, CXCL5, CXCL6, CXCL7, CXCL8, MIF, while the cytokine CCL2 binds to CCR1, CCR2, CCR4, ACKR1, ACKR2, one or more of which can be targeted with inhibitors as potential treatment options. In an aspect, Interleukin-6 (IL-6) inhibitors (e.g., tocilizumab) have been used for the treatment of immune-related adverse events. The elevation of IL-6 in a patient may not only be predictive/diagnostic of irAE but also indicate a treatment. Examples of other potential target cytokines/cytokine receptors and corresponding inhibitors include but are not restricted to CCL2 (inhibitors: bindarit—inhibits CCL2 production, MLN1202—is an anti-CCR2 monoclonal antibody and targets a receptor for CCL2), IL-6 (inbitors: siltuximab—inhibits IL-6, tocilizumab and sarilumab—inhibit IL-6 receptor and dasatinib—inhibits production of IL-6), GM-CSF (inhibitors: gimsilumab or lenzilumab—target GM-CSF directly, mavrilumumab—target GM-CSF receptor), TNF (inhibitors: infliximab, adalimumab, etanercept or dasatinib—inhibits production of TNF) and IL-4 (inhibitor: dupilumab—IL-4 receptor blocker).

In some aspects, a non-ICI therapy, non-limiting examples of which can include chemotherapy, hormonal therapy, small molecule therapy, toxin therapy, prodrug-activating enzyme therapy, biologic therapy, surgical therapy, anti-angiogenic therapy, targeted therapy, epigenetic therapy, demethylation therapy, histone deacetylase inhibitor therapy, differentiation therapy, radiation therapy, stem cell transplantation and/or any combination thereof.

Cancer therapeutic agents or chemotherapeutic agents include alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.RTM.; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb Oncology, Princeton, N.J.) and doxetaxel (TAXOTEPvE™, Pvhne-Poulenc Rorer, Antony, France); chlorambucil; gemcitabine; 6-thioguanine; mercaptopurine; methotrexate; platinum analogs such as cisplatin and carboplatin; vinblastine; trastuzumab, docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as Targretin™ (bexarotene), Panretin™ (alitretinoin); ONTAKT™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 1 17018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer therapeutic agents include sorafenib and other protein kinase inhibitors such as afatinib, axitinib, bevacizumab, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, trastuzumab, vandetanib, vemurafenib, and sunitinib; sirolimus (rapamycin), everolimus and other mTOR inhibitors. Examples of additional chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Exemplary chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase.

In some aspects, modifying an ICI treatment may include recommending a decreased dosing level of an ICI, to be about 10, 20, 30, 40, 50, 60, 70, 80, 90, or 95% less than the typical dose for a particular indication or individual (e.g., about 3 mg/kg, about 2.5 mg/kg, about 2 mg/kg, about 1.5 mg/kg, about 1 mg/kg, about 0.9 mg/kg, about 0.8 mg/kg, about 0.7 mg/kg, about 0.6 mg/kg, about 0.5 mg/kg, about 0.4 mg/kg, about 0.3 mg/kg, about 0.2 mg/kg, about 0.1 mg/kg, about 0.05 mg/kg, about 0.01 mg/kg, about 0.005 mg/kg), or about 1.5×, 2×, 2.5×, 3×, 3.5×, 4×, 4.5×, 5×, 6×, 7×, 8×, 9×, or 10× less ICI than the ICI dose for a particular indication or for individual. Such modification to the ICI treatment may be undertaken either alone or in conjunction with corticosteroids or other cancer treatments.

In another aspect, a reduced frequency of administration of an ICI treatment can be recommended. For example, such a reduced frequency of administration of an ICI treatment may include an administration as follows: about once every 3 weeks, about once every 3.5 weeks, about once every 4 weeks, about once every 4.5 weeks, about once every 5 weeks, about once every 5.5 weeks, about once every 6 weeks, about once every 6.5 weeks, about once every 7 weeks, about once every 7.5 weeks, about once every 8 weeks, about once every 8.5 weeks, about once every 9 weeks, about once every 2.5 months, about once every 3 months, about once every 3.5 months, about once every 4 months, about once every 4.5 months, about once every 5 months, about once every 5.5 months, and about once every 6 months. In some aspects, the reduced frequency of administration may be recommended either alone or in conjunction with corticosteroids or other cancer treatment.

In some aspects, the disclosed methods prevent, reduce, and/or treat one or more ICI associated irAE and/or one or more symptoms associated with irAE. In some aspects, the methods disclosed herein prevent, reduce, and/or treat one or more ICI associated irAE and/or one or more symptoms associated with irAE by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or more.

In some aspects, the method for treating cancer comprises providing a sample from a subject, assessing abundance of one or more immune cell subsets in the sample, treating the subject with an ICI treatment if subject is diagnosed as low risk for irAE if the abundance of one or more immune cells in Table 1 are different in the sample from the subject when compared to the abundance in a control sample. In some aspects, the subject receives a non-ICI treatment, a modified ICI treatment, or an ICI treatment and an irAE mitigating treatment if the subject is diagnosed as high risk if the abundance of at least one immune cell in Table 1 is different in the sample when compared to the abundance in a control sample.

In some aspects of the present disclosure, the method for treating cancer comprises providing a sample from a subject, assessing the abundance of one or more immune cells in the sample, treating the subject with a non-ICI treatment, a modified ICI treatment, or an ICI treatment and an irAE mitigating treatment if subject is diagnosed as high risk for rAE associated with immune checkpoint inhibitor (ICI) treatment, when the immune cell profile comprises one or more cell subsets at a lower abundance before initiation of ICI treatment at baseline, or after ICI treatment in the sample from the subject when compared to a control sample. In some aspects, the one or more immune cell subsets related to irAE associated with ICI treatment that are present at lower abundance before initiation of ICI treatment at baseline, or after ICI treatment can be one or more of naïve B cells, CD57hiCD56+TE CD8 T cells, memory B cells, gamma delta T cells, naïve CD8 T cells, naïve CD4 T cells, or any combination thereof. In some aspects, subject receives a non-ICI treatment, a modified ICI treatment, or an ICI treatment and an irAE mitigating treatment, if the abundance before initiation of ICI treatment at baseline, or after ICI treatment of naïve B cells is lower in the sample from the subject, when compared to the abundance in a control sample. In some aspects, subject receives a non-ICI treatment, a modified ICI treatment, or an ICI treatment and an irAE mitigating treatment, if the abundance before initiation of ICI treatment at baseline, or after ICI treatment of CD57hiCD56+TE CD8 T cells is lower in the sample from the subject, when compared to the abundance in a control sample. In some aspects, subject receives a non-ICI treatment, a modified ICI treatment, or an ICI treatment and an irAE mitigating treatment, if the abundance before initiation of ICI treatment at baseline, or after ICI treatment of memory B cells is lower in the sample from the subject, when compared to the abundance in a control sample. In some aspects, subject receives a non-ICI treatment, a modified ICI treatment, or an ICI treatment and an irAE mitigating treatment, if the abundance before initiation of ICI treatment at baseline, or after ICI treatment of gamma delta T cells is lower in the sample from the subject, when compared to the abundance in a control sample. In some aspects, subject receives a non-ICI treatment, a modified ICI treatment, or an ICI treatment and an irAE mitigating treatment, if the abundance before initiation of ICI treatment at baseline, or after ICI treatment of naïve CD8 T cells is lower in the sample from the subject, when compared to the abundance in a control sample. In some aspects, subject receives a non-ICI treatment, a modified ICI treatment, or an ICI treatment and an irAE mitigating treatment, if the abundance before initiation of ICI treatment at baseline, or after ICI treatment of naïve CD4 T cells is lower in the sample from the subject, when compared to the abundance in a control sample.

In some aspects of the present disclosure, the method for treating cancer comprises providing a sample from a subject, assessing the abundance of one or more immune cells in the sample, treating the subject with an ICI treatment if subject is diagnosed as low risk for irAE associated with immune checkpoint inhibitor (ICI) treatment, when the immune cell profile comprises one or more cell subsets at a higher abundance before initiation of ICI treatment at baseline, or after ICI treatment in the sample from the subject when compared to a control sample. In some aspects, the one or more immune cell subsets related to irAE associated with ICI treatment that are present at higher abundance before initiation of ICI treatment at baseline, or after ICI treatment can be one or more of naïve B cells, CD57hiCD56+TE CD8 T cells, memory B cells, gamma delta T cells, naïve CD8 T cells, naïve CD4 T cells, or any combination thereof. In some aspects, subject receives an ICI treatment, if the abundance before initiation of ICI treatment at baseline, or after ICI treatment of naïve B cells is higher in the sample from the subject, when compared to the abundance in a control sample. In some aspects, subject receives an ICI treatment, if the abundance before initiation of ICI treatment at baseline, or after ICI treatment of CD57hiCD56+TE CD8 T cells is higher in the sample from the subject, when compared to the abundance in a control sample. In some aspects, subject receives an ICI treatment, if the abundance before initiation of ICI treatment at baseline, or after ICI treatment of memory B cells is higher in the sample from the subject, when compared to the abundance in a control sample. In some aspects, subject receives an ICI treatment, if the abundance before initiation of ICI treatment at baseline, or after ICI treatment of gamma delta T cells is higher in the sample from the subject, when compared to the abundance in a control sample. In some aspects, subject receives an ICI treatment, if the abundance before initiation of ICI treatment at baseline, or after ICI treatment of naïve CD8 T cells is higher in the sample from the subject, when compared to the abundance in a control sample. In some aspects, subject receives an ICI treatment, if the abundance before initiation of ICI treatment at baseline, or after ICI treatment of naïve CD4 T cells is higher in the sample from the subject, when compared to the abundance in a control sample.

In some aspects, the method for treating cancer comprises providing a sample from a subject, assessing the abundance of one or more immune cells in the sample, treating the subject with an ICI treatment if subject is diagnosed as low risk for irAE if the abundance of one or more immune cells CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells or any combination thereof, are lower or equal to the abundance in a control sample. In some aspects, the subject receives an ICI treatment if the abundance of effector memory (EM) CD8 T cells is lower or equal in the sample from the subject, when compared to the abundance in a control sample. In some aspects, the subject receives an ICI treatment if the abundance of CD57lo CD56+TE CD8 T cells is lower or equal in the sample from the subject when compared to the abundance in the control sample. In some aspects, the subject receives an ICI treatment if the abundance of plasmablasts is lower or equal in the sample from the subject, when compared to the abundance in a control sample. In some aspects, the subject receives an ICI treatment if the abundance of pDC is lower or equal in the sample from the subject, when compared to the abundance in a control sample. In some aspects, the subject receives an ICI treatment if the abundance of CLTA-4+ naïve B cells is lower or equal in the sample from the subject, when compared to the abundance in a control sample.

In some aspects, the subject receives a non-ICI treatment, a modified ICI treatment, or an ICI treatment and an irAE mitigating treatment if the subject is diagnosed as high risk of irAE if the abundance of at least one or more of CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, or any combination thereof is greater in the sample from the subject compared to the abundance in a control sample. In some aspects, the subject receives a non-ICI treatment, a modified ICI treatment, or an ICI treatment and an irAE mitigating treatment if the abundance of TE CD8 T cells is greater in the sample from the subject compared to the abundance in a control sample. In some aspects, TE CD8 T cells comprise CD57lo CD56− TE CD8 T cells, CD57hi CD56− TE CD8 T cells, or any combination thereof. In some aspects, the subject receives a non-ICI treatment, a modified ICI treatment, or an ICI treatment and an irAE mitigating treatment when the abundance of effector memory (EM) CD8 T cells is higher in the sample from the subject, when compared to the abundance in a control sample. In some aspects, the subject receives a non-ICI treatment, a modified ICI treatment, or an ICI treatment and an irAE mitigating treatment if the abundance of CD57lo CD56+TE CD8 T cells is higher in the sample from the subject when compared to the abundance in the control sample. In some aspects, the subject receives a non-ICI treatment, a modified ICI treatment, or an ICI treatment and an irAE mitigating treatment if the abundance of plasmablasts is higher in the sample from the subject, when compared to the abundance in a control sample. In some aspects, the subject receives a non-ICI treatment, a modified ICI treatment, or an ICI treatment and an irAE mitigating treatment if the abundance of pDC is higher in the sample from the subject, when compared to the abundance in a control sample. In some aspects, the subject receives a non-ICI treatment, a modified ICI treatment, or an ICI treatment and an irAE mitigating treatment if the abundance of CLTA-4+ naïve B cells is higher in the sample from the subject, when compared to the abundance in a control sample.

In some aspects, the method for treating cancer comprises providing a sample from a subject, assessing abundance of one or more immune cells in the sample, treating the subject with an ICI treatment if subject is diagnosed as low risk for irAE if the abundance of one or more gamma delta T cells, naïve CD8 T cells, naïve CD4 T cells, or any combination thereof, is higher in the sample from the subject compared to the abundance in a control sample. In some aspects, the subject receives an ICI treatment, if the abundance of gamma delta T cells is higher in the sample from the subject, when compared to the abundance in a control sample. In some aspects, the subject receives an ICI treatment, if the abundance of naïve CD8 T cells is higher in the sample from the subject, when compared to the abundance in a control sample. In some aspects, the subject receives an ICI treatment, if the abundance of naïve CD4 T cells is higher in the sample from the subject, when compared to the abundance in a control sample.

In some aspects, the subject receives a non-ICI treatment, a modified ICI treatment, or an ICI treatment and an irAE mitigating treatment if the subject is diagnosed as high risk if the abundance of at least one of gamma delta T cells, naïve CD8 T cells, naïve CD4 T cells, or any combination thereof is lower in the sample from the subject when compared to the abundance in a control sample. In some aspects, the subject receives a non-ICI treatment, a modified ICI treatment, or an ICI treatment and an irAE mitigating treatment, if the abundance of gamma delta T cells is lower in the sample from the subject, when compared to the abundance in a control sample. In some aspects, the subject receives a non-ICI treatment, a modified ICI treatment, or an ICI treatment and an irAE mitigating treatment, if the abundance of naïve CD8 T cells is lower in the sample from the subject, when compared to the abundance in a control sample. In some aspects, the subject receives a non-ICI treatment, a modified ICI treatment, or an ICI treatment and an irAE mitigating treatment, if the abundance of naïve CD4 T cells is lower in the sample from the subject, when compared to the abundance in a control sample.

In some aspects, a subject receives a non-ICI treatment, a modified ICI treatment, or an ICI treatment and an irAE mitigating treatment when predicted as having a higher risk for developing and/or diagnosed with irAE associated with ICI treatment, when the immune cell profile comprises one or more immune cell subsets at a greater abundance in the sample from the subject compared to the abundance in a control sample, before the initiation of ICI treatment. In some aspects, the pre-treatment or baseline immune cell subsets when present at higher abundance that is associated with higher risk of developing irAE associated with ICI treatment can comprise one or more of TE CD8 T cells, effector memory (EM) CD8 T cells, CD57lo CD56+TE CD8 T cell, or any combination thereof. In some aspects, the subject receives a non-ICI treatment, a modified ICI treatment, or an ICI treatment and an irAE mitigating treatment, if the abundance of TE CD8 T cells is higher in the sample from the subject, when compared to the abundance in a control sample before the initiation of ICI treatment. In some aspects, the subject receives a non-ICI treatment, a modified ICI treatment, or an ICI treatment and an irAE mitigating treatment, if the abundance of effector memory (EM) CD8 T cells, is higher in the sample from the subject, when compared to the abundance in a control sample before the initiation of ICI treatment. In some aspects, the subject receives a non-ICI treatment, a modified ICI treatment, or an ICI treatment and an irAE mitigating treatment, if the abundance of CD57lo CD56+TE CD8 T cell, is higher in the sample from the subject, when compared to the abundance in a control sample before the initiation of ICI treatment.

In some aspects, a subject receives an ICI treatment when predicted as having a lower risk for developing and/or diagnosed with irAE associated with ICI treatment, when the immune cell profile comprises one or more immune cell subsets at a lower abundance in the sample from the subject compared to the abundance in a control sample, before the initiation of ICI treatment. In some aspects, the pre-treatment or baseline immune cell subsets when present at lower abundance that is associated with lower risk of developing irAE associated with ICI treatment can comprise one or more of TE CD8 T cells, effector memory (EM) CD8 T cells, CD57lo CD56+TE CD8 T cell, or any combination thereof. In some aspects, the subject receives an ICI treatment, if the abundance of TE CD8 T cells is lower in the sample from the subject, when compared to the abundance in a control sample before the initiation of ICI treatment. In some aspects, the subject receives an ICI treatment, if the abundance of effector memory (EM) CD8 T cells, is lower in the sample from the subject, when compared to the abundance in a control sample before the initiation of ICI treatment. In some aspects, the subject receives an ICI treatment, if the abundance of CD57lo CD56+TE CD8 T cell, is lower in the sample from the subject, when compared to the abundance in a control sample before the initiation of ICI treatment.

In some aspects, a subject receives a non-ICI treatment, a modified ICI treatment, or an ICI treatment and an irAE mitigating treatment when predicted as having a higher risk for developing and/or diagnosed with irAE associated with ICI treatment, when the immune cell profile comprises one or more immune cell subsets at a greater abundance in the sample from the subject when compared to a control sample, when assessed during ICI treatment. In some aspects, the one or more immune cell subsets when present at higher abundance that is associated with higher risk of developing irAE, when assessed during ICI treatment can comprise one or more of TE CD8 T cells, effector memory (EM) CD8 T cells, CD57lo CD56+TE CD8 T cells, plasmablasts, CLTA-4+ naïve B cells, or any combination thereof. In some aspects, the subject receives a non-ICI treatment, a modified ICI treatment, or an ICI treatment and an irAE mitigating treatment, if the abundance of TE CD8 T cells, is higher in the sample from the subject, when compared to the abundance in a control sample during ICI treatment. In some aspects, the subject receives a non-ICI treatment, a modified ICI treatment, or an ICI treatment and an irAE mitigating treatment, if the abundance of effector memory (EM) CD8 T cells, is higher in the sample from the subject, when compared to the abundance in a control sample during ICI treatment. In some aspects, the subject receives a non-ICI treatment, a modified ICI treatment, or an ICI treatment and an irAE mitigating treatment, if the abundance of CD57lo CD56+TE CD8 T cells, is higher in the sample from the subject, when compared to the abundance in a control sample during ICI treatment. In some aspects, the subject receives a non-ICI treatment, a modified ICI treatment, or an ICI treatment and an irAE mitigating treatment, if the abundance of plasmablasts, is higher in the sample from the subject, when compared to the abundance in a control sample during ICI treatment. In some aspects, the subject receives a non-ICI treatment, a modified ICI treatment, or an ICI treatment and an irAE mitigating treatment, if the abundance of CLTA-4+ naïve B cells, is higher in the sample from the subject, when compared to the abundance in a control sample during ICI treatment.

In some aspects, a subject receives an ICI treatment when predicted as having a lower risk for developing and/or diagnosed with irAE associated with ICI treatment, when the immune cell profile comprises one or more immune cell subsets at a lower or equal abundance in the sample from the subject when compared to a control sample, when assessed during ICI treatment. In some aspects, the one or more immune cell subsets when present at lower or equal abundance that is associated with higher risk of developing irAE, when assessed during ICI treatment can comprise one or more of TE CD8 T cells, effector memory (EM) CD8 T cells, CD57lo CD56+TE CD8 T cells, plasmablasts, CLTA-4+ naïve B cells, or any combination thereof. In some aspects, the subject receives an ICI treatment, if the abundance of TE CD8 T cells, is lower or equal in the sample from the subject, when compared to the abundance in a control sample during ICI treatment. In some aspects, the subject receives an ICI treatment, if the abundance of effector memory (EM) CD8 T cells, is lower or equal in the sample from the subject, when compared to the abundance in a control sample during ICI treatment. In some aspects, the subject receives an ICI treatment, if the abundance of CD57lo CD56+TE CD8 T cells, is lower or equal in the sample from the subject, when compared to the abundance in a control sample during ICI treatment. In some aspects, the subject receives an ICI treatment, if the abundance of plasmablasts, is lower or equal in the sample from the subject, when compared to the abundance in a control sample during ICI treatment. In some aspects, the subject receives an ICI treatment and an irAE mitigating treatment, if the abundance of CLTA-4+ naïve B cells, is lower or equal in the sample from the subject, when compared to the abundance in a control sample during ICI treatment.

In some aspects the method of treating a subject with cancer comprises providing a sample from a subject, assessing the level of one or more autoantibodies in the sample from the subject, and treating the subject with an ICI treatment if subject is diagnosed as low risk for irAE if the level of one or more autoantibodies in FIG. 2D are lower or equal to the level in a control sample. In some aspects, the subject receives a non-ICI treatment, a modified ICI treatment, or an ICI treatment and an irAE mitigating treatment if the subject is diagnosed as high risk if the level of one or more autoantibodies in FIG. 2D are greater in the sample from the subject when compared to the level in a control sample.

In some aspects the method of treating a subject with cancer comprises providing a sample from a subject, assessing the level of one or more autoantibodies in the sample, treating the subject with an ICI treatment if subject is diagnosed as low risk for irAE if the level of one or more of AGTR, α-actin, U1-snRNP A, amyloid, collagen II, or any combination thereof, are lower or equal in the sample from the subject when compared to the level in a control sample. In some aspects, the subject receives an ICI treatment if the level of AGTR is lower or equal in the sample from the subject when compared to the level in a control sample. In some aspects, the subject receives an ICI treatment if the level of α-actin is lower or equal in the sample from the subject when compared to the level in a control sample. In some aspects, the subject receives an ICI treatment if the level of U1-snRNP A is lower or equal in the sample from the subject when compared to the level in a control sample. In some aspects, the subject receives an ICI treatment if the level of amyloid is lower or equal in the sample from the subject when compared to the level in a control sample. In some aspects, the subject receives an ICI treatment if the level of collagen II is lower or equal in the sample from the subject when compared to the level in a control sample.

In some aspects, the subject receives a non-ICI treatment, a modified ICI treatment, or an ICI treatment and an irAE mitigating treatment if the subject is diagnosed as high risk if the level of one or more of AGTR, α-actin, U1-snRNP A, amyloid, collagen II, or any combination thereof, are greater in the sample from the subject when compared to the level in a control sample. In some aspects, the subject receives a non-ICI treatment, a modified ICI treatment, or an ICI treatment and an irAE mitigating treatment if the level of AGTR is greater in the sample from the subject when compared to the level in a control sample. In some aspects, the subject receives a non-ICI treatment, a modified ICI treatment, or an ICI treatment and an irAE mitigating treatment if the level of α-actin is greater in the sample from the subject when compared to the level in a control sample. In some aspects, the subject receives a non-ICI treatment, a modified ICI treatment, or an ICI treatment and an irAE mitigating treatment if the level of U1-snRNP A is greater in the sample from the subject when compared to the level in a control sample. In some aspects, the subject receives a non-ICI treatment, a modified ICI treatment, or an ICI treatment and an irAE mitigating treatment if the level of amyloid is greater in the sample from the subject when compared to the level in a control sample. In some aspects, the subject receives a non-ICI treatment, a modified ICI treatment, or an ICI treatment and an irAE mitigating treatment if the level of collagen II is greater in the sample from the subject when compared to the level in a control sample.

In some aspects the method of treating a subject with cancer comprises providing a sample from a subject, assessing the expression of one or more cytokines in the sample, treating the subject with an ICI treatment if subject is diagnosed as low risk for irAE if the expression of one or more cytokines in FIG. 2A or FIG. 2B are lower or equal in the sample from the subject compared to the expression in a control sample. In some aspects, the subject receives a non-ICI treatment, a modified ICI treatment, or an ICI treatment and an irAE mitigating treatment if the subject is diagnosed as high risk if the expression of one or more cytokines in FIG. 2A or FIG. 2B are greater in the sample from the subject compared to the expression in a control sample.

In some aspects the method of treating a subject with cancer comprises providing a sample from a subject, assessing expression of one or more cytokines in the sample, treating the subject with an ICI treatment if subject is diagnosed as low risk for irAE if the expression of one or more of IL-6, IL-1β, TNFα, IFNγ, CXCL2, CCL17, or any combination thereof, are lower or equal in the sample from the subject when compared to the expression in a control sample. In some aspects, the subject receives an ICI treatment if the expression of IL-6 is lower or equal in the sample from the subject when compared to the expression in a control sample. In some aspects, the subject receives an ICI treatment if the expression of IL-1β is lower or equal in the sample from the subject when compared to the expression in a control sample. In some aspects, the subject receives an ICI treatment if the expression of TNFα is lower or equal in the sample from the subject when compared to the expression in a control sample. In some aspects, the subject receives an ICI treatment if the expression of IFNγ is lower or equal in the sample from the subject when compared to the expression in a control sample. In some aspects, the subject receives an ICI treatment if the expression of CXCL2 is lower or equal in the sample from the subject when compared to the expression in a control sample. In some aspects, the subject receives an ICI treatment if the expression of CCL17 is lower or equal in the sample from the subject when compared to the expression in a control sample.

In some aspects, the subject receives a non-ICI treatment, a modified ICI treatment, or an ICI treatment and an irAE mitigating treatment if the subject is diagnosed as high risk if the expression of one or more of IL-6, IL-1β, TNFα, IFNγ, CXCL2, CCL17, or any combination thereof, are greater in the sample from the subject when compared to the expression in a control sample. In some aspects, the subject receives a non-ICI treatment, a modified ICI treatment, or an ICI treatment and an irAE mitigating treatment if the expression of IL-6 is higher in the sample from the subject when compared to the expression in a control sample. In some aspects, the subject receives a non-ICI treatment, a modified ICI treatment, or an ICI treatment and an irAE mitigating treatment if the expression of IL-1β is higher in the sample from the subject when compared to the expression in a control sample. In some aspects, the subject receives a non-ICI treatment, a modified ICI treatment, or an ICI treatment and an irAE mitigating treatment if the expression of TNFα is higher in the sample from the subject when compared to the expression in a control sample. In some aspects, the subject receives a non-ICI treatment, a modified ICI treatment, or an ICI treatment and an irAE mitigating treatment if the expression of IFNγ is higher in the sample from the subject when compared to the expression in a control sample. In some aspects, the subject receives a non-ICI treatment, a modified ICI treatment, or an ICI treatment and an irAE mitigating treatment if the expression of CXCL2 is higher in the sample from the subject when compared to the expression in a control sample. In some aspects, the subject receives a non-ICI treatment, a modified ICI treatment, or an ICI treatment and an irAE mitigating treatment if the expression of CCL17 is higher in the sample from the subject when compared to the expression in a control sample.

The present disclosure further provides a method of treating a subject with cancer comprising assessing abundance, level, or expression of one or more of the immune cell subsets, autoantibodies, cytokines, or any combination thereof and assigning a risk level for developing irAE. In some aspects, risk assessment can be done before, during and/or after ICI treatment. In some aspects, the risk assessment comprises predicting, diagnosing, or monitoring ICI-related ocular toxicity. If the profile correlates with irAE or future development of irAE, the subjects are assigned a high risk for developing irAE. If the profile correlates with no irAE the subjects are assigned as low risk for developing irAE.

In some aspects the method of treating a subject with cancer further comprises providing a sample from a subject, assessing immune profile, autoantibody profile, cytokine profile, or any combination thereof, the sample. The profile comprises one or more of immune cells, autoantibodies, cytokines, provided in Table 2, FIG. 3F, Table 4, FIG. 2D, FIG. 2A, FIG. 2B respectively, or any combination thereof. The method further comprises treating the subject with an ICI treatment if subject is diagnosed as low risk for irAE if, the abundance of one or more immune cell subsets in Table 1, FIG. 3F, Table 4, the level of one or more autoantibodies in FIG. 2D, the expression of one or more cytokines in FIG. 2A or FIG. 2B, are at lower or equal in the sample from the subject when compared to the abundance, level, or expression in a control sample, or any combination thereof and/or if the if the abundance of one or more immune cells gamma delta T cells, naïve CD8 T cells, naïve CD4 T cells, or any combination thereof, are higher in the sample from the subject when compared to the abundance in a control sample. In some aspects, the subject receives a non-ICI treatment, a modified ICI treatment, or an ICI treatment and an irAE mitigating treatment if the subject is diagnosed as high risk if the abundance of one or more immune cells in Table 1, FIG. 3F, Table 4, the level of one or more autoantibodies in FIG. 2D, the expression of one or more cytokines in FIG. 2A or FIG. 2B, are higher in the sample from the subject when compared to a control sample, or any combination thereof, and/or if the abundance of one or more immune cells gamma delta T cells, naïve CD8 T cells and/or naïve CD4 T cells, or any combination thereof, are lower in the sample from the subject when compared to abundance in a control sample.

In some aspects the method of treating a subject with cancer comprises providing a sample from a subject, assessing immune profile, autoantibody profile, cytokine profile, or any combination thereof, the sample. The profile comprises one or more of CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, gamma delta T cells, naïve CD8 T cells, naïve CD4 T cells, angiotensin receptor (AGTR), α-actin, U1-snRNP A, amyloid, collagen II, BAFF, elastin, Collagen I, Histone H3, Proteoglycan, EBNA1, DGPS, MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-α, or any combination thereof. The method further comprises treating the subject with an ICI treatment if subject is diagnosed as low risk for irAE if, the abundance, level, or expression of one or more of CD57lo CD56− TE CD8 T cells, CD57hi CD56− TE CD8 T cells, effector memory (EM) CD8 T cell, CD57lo CD56+TE CD8 T cell, plasmablasts, pDC, CLTA-4+ naïve B cells, AGTR, α-actin, U1-snRNPA, amyloid, collagen II, BAFF, elastin, collagen I, Histone H3, proteoglycan, EBNA1, DGPS, MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-α, or any combination thereof, are at lower or equal in the sample from the subject, when compared to the abundance, level, or expression in a control sample, or any combination thereof and/or if the if the abundance of one or more of gamma delta T cells, naïve CD8 T cells, naïve CD4 T cells, or any combination thereof, are higher in the sample from the subject when compared to the abundance in a control sample. In some aspects, the subject receives a non-ICI treatment, a modified ICI treatment, or an ICI treatment and an irAE mitigating treatment if the subject is diagnosed as high risk if the abundance, level, or expression of one or more of CD57lo CD56− TE CD8 T cells, CD57hi CD56− TE CD8 T cells, effector memory (EM) CD8 T cell, CD57lo CD56+TE CD8 T cell, plasmablasts, pDC, CLTA-4+naïve B cells, AGTR, α-actin, U1-snRNP A, amyloid, collagen 1l, BAFF, elastin, collagen I, Histone H3, Proteoglycan, EBNA1, DGPS, MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-α, or any combination thereof, are higher in the sample from the subject, when compared to abundance, level, or expression in a control sample, or any combination thereof, and/or if the if the abundance of one or more of gamma delta T cells, naïve CD8 T cells, naïve CD4 T cells, or any combination thereof, are lower in the sample from the subject when compared to the abundance in a control sample.

In some aspects the method of treating a subject with cancer comprises providing a sample from a subject, assessing immune profile, autoantibody profile, cytokine profile, or any combination thereof, the sample. The profile comprises one or more of CD57lo CD56− TE CD8 T cells, CD57hi CD56− TE CD8 T cells, effector memory (EM) CD8 T cell, CD57lo CD56+TE CD8 T cell, CD27− plasmablasts, pDC, CLTA-4+ naïve B cells, gamma delta T cells, naïve CD8 T cells, naïve CD4 T cells, angiotensin receptor (AGTR), α-actin, U1-snRNP A, amyloid, collagen 1l, GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof. The method further comprises treating the subject with an ICI treatment if subject is diagnosed as low risk for irAE if, the abundance, level, or expression of one or more of CD57lo CD56− TE CD8 T cells, CD57hi CD56− TE CD8 T cells, effector memory (EM) CD8 T cell, CD57lo CD56+TE CD8 T cell, plasmablasts, pDC, CLTA-4+ naïve B cells, angiotensin receptor (AGTR), α-actin, U1-snRNP A, amyloid, collagen II, GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof, are lower or equal in the sample from the subject, when compared to the abundance, level, or expression in a control sample, and/or if the if the abundance of one or more of gamma delta T cells, naïve CD8 T cells and/or naïve CD4 T cells, or any combination thereof, are higher in the sample from the subject when compared to the abundance in a control sample. In some aspects, the subject receives a non-ICI treatment, a modified ICI treatment, or an ICI treatment and an irAE mitigating treatment if the subject is diagnosed as high risk if the abundance, level, or expression of one or more of CD57lo CD56− TE CD8 T cells, CD57hi CD56− TE CD8 T cells, effector memory (EM) CD8 T cell, CD57lo CD56+TE CD8 T cell, CD27− plasmablasts, pDC, CLTA-4+ naïve B cells, angiotensin receptor (AGTR), α-actin, U1-snRNP A, amyloid, collagen 1l, GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof, is higher in the sample from the subject, when compared to the abundance, level, or expression in a control sample, or any combination thereof, and/or if the if the abundance of one or more of gamma delta T cells, naïve CD8 T cells, naïve CD4 T cells, or any combination thereof, are lower in the sample from the subject when compared to the abundance in a control sample.

In some aspects the method of treating a subject with cancer comprises providing a sample from a subject, assessing immune profile, autoantibody profile, cytokine profile, or any combination thereof, the sample. The profile comprises one or more of CD57lo CD56− TE CD8 T cells, CD57hi CD56− TE CD8 T cells, effector memory (EM) CD8 T cell, CD57lo CD56+TE CD8 T cell, plasmablasts, pDC, CLTA-4+ naïve B cells, gamma delta T cells, naïve CD8 T cells, naïve CD4 T cells, angiotensin receptor (AGTR), α-actin, U1-snRNP A, amyloid, collagen II, IL-6, IL-1β, TNFα, IFNg, CXCL2, CCL17, or any combination thereof. The method further comprises treating the subject with an ICI treatment if the subject is diagnosed as low risk for irAE when, the abundance, level, or expression of one or more of CD57loCD56− TE CD8 T cells, CD57hi CD56− TE CD8 T cells, effector memory (EM) CD8 T cell, CD57lo CD56+TE CD8 T cell, plasmablasts, pDC, CLTA-4+ naïve B cells, AGTR, α-actin, U1-snRNP A, amyloid, collagen 1l, IL-6, IL-1β, TNFα, IFNg, CXCL2, CCL17, or any combination thereof, are lower or equal in the sample from the subject, when compared to the abundance, level, or expression in a control sample, or any combination thereof, and/or if the abundance of one or more of gamma delta T cells, naïve CD8 T cells, naïve CD4 T cells, or any combination thereof, are higher in the sample from the subject when compared to the abundance in a control sample. In some aspects, the subject receives a non-ICI treatment, a modified ICI treatment, or an ICI treatment and an irAE mitigating treatment if the subject is diagnosed as high risk if the abundance, level, or expression of one or more of CD57lo CD56− TE CD8 T cells, CD57hi CD56− TE CD8 T cells, effector memory (EM) CD8 T cell, CD57lo CD56+TE CD8 T cell, plasmablasts, pDC, CLTA-4+ naïve B cells, angiotensin receptor (AGTR), α-actin, U1-snRNP A, amyloid, collagen II, IL-6, IL-1β, TNFα, IFNg, CXCL2, CCL17, or any combination thereof, are higher in the sample from the subject, when compared to a the abundance, level, or expression in a control sample, or any combination thereof, and/or if the if the abundance of one or more of gamma delta T cells, naïve CD8 T cells and/or naïve CD4 T cells, or any combination thereof, are lower in the sample from the subject when compared to the abundance in a control sample.

In some aspects, irAE can comprise ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof. In some aspects, ocular toxicity comprise uveitis, dry eye syndrome, ocular myasthenia, myositis, ophthalmoplegia, ptosis, Sjogren's, metastases to the vitreous, vasculitis, damage to rod/cone function, macular edema, retinopathy, choroidal neovascularization, chorioretinal atrophy, vascular leakage, choroidal sarcoid granulomas, choroidal detachment, development of subretinal fluid, optic neuritis, nerve palsies associated with eye, arteritic ischemic optic neuropathy, eye inflammation, or any combination thereof. In some aspects, the irAE mitigating therapy can comprise one or more irAE mitigating therapy disclosed herein.

In some aspects, the treatment can comprise inhibition of cancer progression, inhibition of an increase in tumor volume and/or tumor growth, a reduction in tumor volume, size and/or metastasis, a reduction in tumor growth rate, an eradication of a tumor and/or cancer cell, or any combination thereof. The method may also result in a prolonging survival of a subject and/or better prognosis. One skilled in the art can use any known methods for assessing cancer progression, metastases, tumor volume, tumor growth, and/or survival, non-limiting examples of which are physical exams, imaging tests (x-rays, CT scans, etc.), endoscopy exams, and biopsies. In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In some aspects, the disclosed method for treatment using profile comprising immune cell, autoantibody, cytokine, or any combination thereof, can further comprise repeating one or more steps of the disclosed assessment and/or modifying one or more steps of the disclosed treatment. In an aspect, the disclosed method can further comprise of modifying one or more of the administrations of the ICI compounds. In some aspects the method, can comprise modifying one or more of steps of administration. For example, in an aspect, the method can be altered by changing the amount of one or more of the ICI compounds, thereof administered to a subject, or by changing the frequency of administration of one or more of the ICI compounds thereof to a subject, or by changing the duration of time one or more of the ICI compounds administered to a subject. In some aspects, the ICI compounds comprise one or more ICI compounds disclosed herein In some aspects, the assessment using profile comprising immune cell, autoantibody, cytokine, or any combination thereof, can be repeated one or more times at the start of the treatment. In some aspects, the assessment using profile comprising immune cell, autoantibody, cytokine, or any combination thereof, can be repeated one or more times during the treatment. In some aspects, the assessment using profile comprising immune cell, autoantibody, cytokine, or any combination thereof, can be repeated one or more times after the treatment is completed.

In some aspects, the assessments can be automated using computer software analytical programs. The present disclosure provides computer implemented methods of detaining, comparing, and analyzing patterns of abundance or expression of one or more immune cell, autoantibody, cytokine or any combination thereof, in order to assess the risk of irAE in a subject. The analytical programs can be interfaced with, for example, programs that are part of an automated autoantibodies, cytokines and/or immune cell detection or quantification system so that data from the automated detection or quantification system can fed directly to the analytical programs. Computer implemented programs can be implemented to output, for example, the identity of immune cells, autoantibodies, and/or cytokines in the sample and the degree of upregulation or downregulation. The interface between the analytical programs may be direct or indirect. In some aspects, the programs of this disclosure can be designed to accept information on the detection or quantification of one or more immune cell, autoantibody, and/or cytokine, are able to implement data analysis, and output risk assessments. In some aspects the programs of disclosure can further output treatment strategies.

### IV. Methods of Identifying Differential Expression of Biomarkers Associated with IRAE

In some aspects, the present disclosure provide a method of identifying the presence of at least one differentially abundant immune cell subsets associated with irAE in a biological sample of a subject with cancer. The method comprises providing a sample from the subject and assessing one or more immune cells in said sample, wherein the assessment comprises detecting one or more immune cell subsets disclosed in Table 1. The disclosed assessment can be conducted in a subject who is planning, undergoing, or has completed ICI treatment.

In some aspects, the method of identifying differentially abundant immune cell subsets can comprise identifying one or more immune cell subsets associated with a baseline or a pre-treatment immune cell profile that correlates with future irAE. In some aspects, the disclosed method can identify an immune cell subset which comprises an interim immune cell profile that correlates with ICI treatment before the onset of irAE. In some aspects, the ICI treatment is an ICI treatment disclosed herein. In some aspects, the disclosed method can identify an immune cell profile which can comprise one or more immune cell subsets that correlates with onset of irAE during treatment. In some aspects, the disclosed method can identify one or more immune cell subsets which can have elevated abundance which appear at baseline or pre-treatment and sustain during irAE. In some aspects, the disclosed method can identify one or more immune cell subsets which can have elevated abundance after onset irAE or during later part of the ICI treatment. In some aspects, the elevated abundance of one or more identified immune cell can reduce in the subject after treatment with irAE mitigating therapy.

In some aspects, if the identification detects one or more immune cell subsets disclosed in Table 1 in the subject with an abundance that is different in a sample from the subject when compared to the abundance in a control sample, then the subject receives an ICI treatment and an irAE mitigating treatment as disclosed herein. In some aspects, if the identification detects one or more immune cells disclosed in Table 1 in the sample from the subject at a similar abundance compared to abundance in a control sample, then the subject receives an ICI treatment.

In some aspects, the detection of one or more immune cell can be combined with detection of one or more cytokines and/or one or more autoantibodies.

In some aspects, one or more immune cell subsets related to irAE associated with ICI treatment that can be detected at lower abundance compared to control subject with no irAE or a healthy subject, and can comprise one or more of gamma delta T cell, naïve CD8 T cell, naïve CD4 T cell, or any combination thereof. In such cases, a subject can be predicted as having a higher risk for irAE associated with ICI treatment. In some aspects, the one or more immune cell subsets related to irAE associated with ICI treatment that is detected at lower abundance comprises gamma delta T cell subset. In some aspects, the one or more immune cell subsets related to irAE associated with ICI treatment that is detected at lower abundance comprise a naïve CD8 T cell subset. In some aspects, the one or more immune cell subsets related to irAE associated with ICI treatment that is detected at lower abundance comprise a naïve CD4 T cell subset.

In some aspects, one or more immune cell related to irAE associated with ICI treatment that can be detected at a higher abundance in the sample from the subject when compared to a control sample. In some aspects, the pre-treatment or baseline immune cell subsets when detected at higher abundance that is associated with higher risk of developing irAE associated with ICI treatment can comprise one or more of TE CD8 T cells, effector memory (EM) CD8 T cells, CD57lo CD56+TE CD8 T cells, or any combination thereof. In some aspects, the pre-treatment or baseline immune cell subsets when present at higher abundance that is associated with higher risk of developing irAE associated with ICI treatment can comprise TE CD8 T cells. In some aspects, the TE CD8 T cells can comprise CD57lo CD56− TE CD8 T cells CD57hi CD56− TE CD8 T cells, or any combination thereof. In some aspects, the pre-treatment or baseline immune cell subsets when detected at higher abundance that is associated with higher risk of developing irAE associated with ICI treatment can comprise an effector memory (EM) CD8 T cells. In some aspects, the pre-treatment or baseline immune cell subsets when detected at higher abundance that is associated with higher risk of developing irAE associated with ICI treatment can comprise CD57lo CD56+TE CD8 T cells.

In some aspects, the one or more immune cell subsets when detected at higher abundance that is associated with higher risk of developing irAE, when assessed during ICI treatment can comprise one or more of TE CD8 T cells, effector memory (EM) CD8 T cells, CD57lo CD56+TE CD8 T cells, plasmablasts, CLTA-4+ naïve B cells, or any combination thereof. In some aspects, the one or more immune cell subsets when detected at higher abundance that is associated with higher risk of developing irAE, when assessed during ICI treatment can comprise TE CD8 T cells. In some aspects, the TE CD8 T cells can comprise CD57lo CD56− TE CD8 T cells, CD57hi CD56− TE CD8 T cells, or any combination thereof. In some aspects, the one or more immune cell subsets when detected at higher abundance that is associated with higher risk of developing irAE, when assessed during ICI treatment can comprise CD57lo CD56+TE CD8 T cells. In some aspects, the one or more immune cell subsets when detected at higher abundance that is associated with higher risk of developing irAE, when assessed during ICI treatment can comprise plasmablasts. In some aspects, plasmablasts can comprise plasmablasts, pDC, or any combination thereof. In some aspects, the one or more immune cell subsets when detected at higher abundance that is associated with higher risk of developing irAE, when assessed during ICI treatment can comprise CLTA-4+ naïve B cells.

In some aspects, the present disclosure provide a method of identifying at least one differentially expressed autoantibody associated with irAE in a biological sample of a subject with cancer. The method comprises providing a sample from the subject and assessing one or more autoantibodies in the sample from a subject, wherein the assessment comprises detecting one or more autoantibodies disclosed in FIG. 2D. The disclosed identification can be conducted in a subject who is planning, undergoing, and/or has completed ICI treatment.

In some aspects, method of identifying differentially expressed autoantibody can comprise identifying one or more autoantibodies associated with a baseline or a pre-treatment autoantibody profile that correlates with future irAE. In some aspects, the disclosed method can identify autoantibody profile which comprises an interim autoantibody profile that correlates with ICI treatment before the onset of irAE. In some aspects, the disclosed method can identify an autoantibody profile which can comprise one or more autoantibodies that correlates with onset of irAE during treatment. In some aspects, the disclosed method can identify one or more autoantibodies which can have elevated level which appear at baseline or pre-treatment and sustain during irAE. In some aspects, the disclosed method can identify one or more autoantibodies which can have elevated level after onset irAE or during later part of the ICI treatment. In some aspects, the elevated level of one or more identified autoantibodies can reduce in the subject after treatment with irAE mitigating therapy.

In some aspects, the detection of one or more autoantibodies can be combined with detection of one or more immune cells and/or one or more cytokines.

In some aspects, if the identification detects one or more autoantibodies disclosed in FIG. 2D in the subject at a different level in a sample from the subject when compared to the level in a control sample, then the subject receives an ICI treatment and an irAE mitigating treatment. In some aspects, if the identification detects one or more autoantibodies disclosed in FIG. 2D in the subject at a similar level in the sample from the subject when compared to level in a as control sample, then the subject receives an ICI treatment.

In some aspects, the disclosed autoantibody can comprise one or more of AGTR, α-actin, U1-snRNP A, amyloid, collagen II, BAFF, elastin, collagen I, histone H3, proteoglycan, EBNA1, DGPS, or any combination thereof. In some aspects, the disclosed autoantibody is identified at a higher level in a sample from the subject when compared to a control sample. In some aspects, the autoantibody that can be present at a higher level comprise of AGTR, α-actin, U1-snRNP A, amyloid, collagen II, or any combination thereof. In some aspects, the autoantibody comprises AGTR. In some aspects, the autoantibody comprises α-actin. In some aspects, the autoantibody comprises U1-snRNP A. In some aspects, the autoantibody comprises amyloid. In some aspects, the autoantibody comprises collagen II. In some aspect, the autoantibody comprises BAFF. In some aspect, the autoantibody comprises elastin. In some aspect, the autoantibody comprises collagen I. In some aspect, the autoantibody comprises histone H3. In some aspect, the autoantibody comprises Proteoglycan. In some aspect, the autoantibody comprises EBNA1. In some aspect, the autoantibody comprises DGPS.

In some aspects, the present disclosure provide a method of identifying the presence of at least one differentially expressed cytokine associated with irAE in a biological sample of a subject with cancer. The method comprises providing a sample from the subject and assessing expression of one or more cytokines in the sample, wherein the assessment comprises detecting expression of one or more cytokines disclosed in FIG. 2A, FIG. 2B, or any combination thereof. The disclosed identification can be conducted in a subject who is planning, undergoing, and/or has completed ICI treatment.

In some aspects, method of identifying differentially expressed cytokine can comprise identifying one or more cytokines associated with a baseline or a pre-treatment immune cell profile that correlates with future irAE. In some aspects, the disclosed method can identify a cytokine profile which comprises an interim cytokine profile that correlates with ICI treatment before the onset of irAE. In some aspects, the disclosed method can identify a cytokine profile which can comprise one or more cytokines that correlates with onset of irAE during treatment. In some aspects, the disclosed method can identify one or more cytokines which can have elevated expression which appear at baseline or pre-treatment and sustain during irAE. In some aspects, the disclosed method can identify one or more cytokines which can have elevated expression after onset irAE or during later part of the ICI treatment. In some aspects, the elevated expression of one or more identified cytokines can reduce in the subject after treatment with irAE mitigating therapy.

In some aspects, if the identification detects one or more cytokines disclosed in FIG. 2A, FIG. 2B, or any combination thereof in the subject at a different expression in the sample from the subject when compared to expression in than a control sample, then the subject receives an ICI treatment and an irAE mitigating treatment. In some aspects, if the identification detects expression of one or more cytokines disclosed in FIG. 2A, FIG. 2B, or any combination thereof in the sample from the subject at a similar expression when compared to expression in a control sample, then the subject receives an ICI treatment.

In some aspects, the detection of one or more cytokines can be combined with detection of one or more of immune cells and/or autoantibodies.

In some aspects, identification comprise detecting the expression of MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-a, or any combination thereof. In some aspects, the disclosed cytokine comprise GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof. In some aspects, the disclosed cytokine comprise IL-6, IL-1β, TNFα, IFNγ, CXCL2, CCL17, or any combination thereof.

In some aspects, the present disclosure provide a method of identifying the presence of at least one differentially abundant, level, or expressed immune cell subsets, autoantibodies, and/or cytokines associated with irAE in a biological sample of a subject with cancer. The method comprises providing a sample from the subject and assessing one or more immune cells, one or more autoantibodies and/or one or more cytokines in the sample from the subject, wherein the assessment comprises detecting abundance, level, or expression of one or more immune cells disclosed in Table 1, FIG. 3F, and/or Table 4, one or more autoantibodies in FIG. 2D, and/or one or more cytokines in FIG. 2A, and/or FIG. 2B. The disclosed identification can be conducted in a subject who is planning, undergoing, or has completed ICI treatment.

In some aspects, method of identifying differentially abundant, level, or expressed immune cell subsets, autoantibodies, and/or cytokines can comprise identifying one or more immune cells associated with a baseline or a pre-treatment immune cell subset, one or more autoantibodies, and/or one or more cytokines that correlates with future irAE. In some aspects, the disclosed method can identify one or more immune cells, one or more autoantibodies, and/or one or more cytokines which comprises an interim immune cell, autoantibody, and/or cytokine profile that correlates with ICI treatment before the onset of irAE. In some aspects, the disclosed method can identify an immune cell, autoantibody, and/or cytokine profile which can comprise one or more immune cells, one or more autoantibodies, and/or one or more cytokines that correlates with onset of irAE during treatment. In some aspects, the disclosed method can identify one or more immune cell subsets, one or more autoantibodies, and/or one or more cytokines which can have elevated abundance, level, or expression which appear at baseline or pre-treatment and sustain during irAE. In some aspects, the disclosed method can identify one or more immune cells, one or more autoantibodies, and/or one or more cytokines which can have elevated abundance, level, or expression after onset irAE or during later part of the ICI treatment. In some aspects, the elevated abundance, level, or expression of one or more identified immune cells, autoantibodies, and/or cytokines can reduce in the subject after treatment with irAE mitigating therapy.

In some aspects, if the identification detects abundance of one or more immune cells disclosed in Table 1, FIG. 3F, and/or Table 4, level of one or more autoantibodies in FIG. 2D and/or expression of one or more cytokines in FIG. 2A and/or FIG. 2B in the sample from the subject at a different abundance, level, or expression compared to the abundance, level, or expression in a control sample, then the subject receives an ICI treatment and an irAE mitigating treatment. In some aspects, if the identification detects one or more immune cell disclosed in Table 1, FIG. 3F and/or Table 4, one or more autoantibody in FIG. 2D and/or one or more cytokine in FIG. 2A and/or FIG. 2B in the subject at a similar abundance, level or expression in the sample from the subject, compared to the abundance, level, or expression in a control sample, then the subject receives an ICI treatment.

In some aspects, identification comprises detection of CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, AGTR, α-actin, U1-snRNP A, amyloid, collagen II, BAFF, elastin, Collagen I, Histone H3, Proteoglycan, EBNA1, DGPS, MIF, MCP-2/CCL8, SDF-1a+b/CXCL12, MDC/CCL22, IL-4, MCP-4/CCL13, GM-CSF, Gro-b/CXCL2, TARC/CCL17, IL-6, IFN-g, IL-10, GCP-2/CXC16, I-TAC/CXCL11, Gr-2/CXCL1, IL-1b, Eotaxin-3/CCL26, MCP-3/CCL7, IL-2, MIP-1a/CCL3, Fractalkine/CX3CL1, MIP-3b/CCL19, 1-209/CCL1, TECK/CCL25, SCYB16/CXCL16, MIP-1d/CCL15, MIG/CXCL9, 6Ckine/CCL21, Eotaxin-2/CCL24, IP-10/CXCL10, CTACK/CCL27, MCP-1/CCL2, IL-8/CXCL8, MIP-3a/CCL20, ENA-78/CXCL5, IL-16, MPIF-1/CCL23, BCA-1/CXCL13, Eotaxin/CCL11, TNF-α, or any combination thereof. In some aspects, the disclosed identification comprises detection of CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, AGTR, α-actin, U1-snRNP A, amyloid, collagen II, GM-CSF, Gro-b/CXCL2, TARC/CCL17, IL-6, IFN-g, IL-10, GCP-2/CXCL6, IL-1b, I-TAC/CXCL11, Gro-a/CXCL1, Eotaxin-3/CCL26, MCP-3/CCL7, I-309/CCL1, TECK/CCL25, CTACK/CCL27, IL-16, IP-10/CXCL10, 6Ckine/CCL21, BCA-1/CXCL13, Eotaxin/CCL11, TNF-a, ENA-78/CXCL5, or any combination thereof. In some aspects, the disclosed identification comprise detection of CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, AGTR, α-actin, U1-snRNP A, amyloid, collagen II, IL-6, IL-1β, TNFα, IFNg, CXCL2, CCL17, or any combination thereof. In some aspects, the disclosed identification comprise detection of CD57lo CD56− TE CD8 T cells, CD57hi CD56− TE CD8 T cells, effector memory (EM) CD8 T cell, CD57lo CD56+TE CD8 T cells, plasmablasts, pDC, CLTA-4+ naïve B cells, AGTR, α-actin, U1-snRNP A, amyloid, collagen II, IL-6, IL-1R, TNFα, IFNg, CXCL2, and CCL17.

In some aspects, the disclosed detection of immune cell, autoantibody, cytokine, or any combination thereof, can further comprise repeating one or more steps of the disclosed detection and/or modifying one or more steps of the disclosed treatment. In an aspect, the disclosed method can further comprise of modifying one or more of the administrations of the ICI compounds. In some aspects the method, can comprise modifying one or more of steps of administration. For example, in an aspect, the method can be altered by changing the amount of one or more of the ICI compounds, thereof administered to a subject, or by changing the frequency of administration of one or more of the ICI compounds thereof to a subject, or by changing the duration of time one or more of the ICI compounds administered to a subject. In some aspects, the ICI compounds comprise one or more ICI compounds disclosed herein. In some aspects, the disclosed identification comprising detection of immune cell, autoantibody, cytokine, or any combination thereof, can be repeated one or more times at the start of the treatment. In some aspects, the disclosed identification comprising detection of immune cell, autoantibody, cytokine, or any combination thereof, can be repeated one or more times during the treatment. In some aspects, the disclosed detection of immune cell, autoantibody, cytokine, or any combination thereof, can be repeated one or more times after the treatment is completed.

### V. Kits

The present disclosure provides kits for use in assessing risk of irAE and/or treating cancer, as described herein. Such kits can include one or more containers comprising ready-to-use microarray chips, or other detection devices, computer software data analysis for assessing risk and determining treatment strategies. In some aspects, kits can further include one or more containers comprising active agents for treatment of cancer or mitigating the irAE.

Having described several aspects, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Patient, Materials, and Methods

### Clinical Data and Sample Collection

The patient was enrolled in a prospective biospecimen collection protocol approved by the UT Southwestern IRB (#STU 082015-053). The patient provided written, informed consent for additional data collection, presentation, and publication. Clinical, radiographic, and laboratory data were collected from the Epic electronic health record (Epic, Verona, Wisconsin).

Peripheral blood samples were examined from the patient and two cases without irAE (no toxicity, NT) at pre-ICI baseline, two weeks, six weeks, three months, every three months thereafter, and after initiation of steroids for irAE treatment. Serial samples (baseline, two weeks) from two healthy controls were also examined. Samples were centrifuged at 3000 rpm at 4° C. for 15 min to obtain plasma. Peripheral blood mononuclear cells (PBMC) were isolated from samples using density gradient centrifugation in Ficoll-Paque PLUS Media (Fisher Scientific, Waltham, MA).

### Cytokine/Chemokine Analysis

Monitoring of cytokine and chemokine levels was performed using Bio-Plex Pro Human Chemokine 40-plex Panel (Bio-Rad Laboratories, Hercules, California) using a Luminex 200 System. Bio-Plex Manager™ 6.1 software was used for data analysis. Concentrations of cytokines and chemokines (pg/mL) were determined on the basis of the fit of a standard curve for mean fluorescence intensity versus pg/mL. These cytokines are stable over time in healthy controls not receiving ICI. Heatmaps were generated using Genesis cluster analysis of microarray data.

### Autoantigen Array Analysis

Serum autoantibody profiling was performed using a custom protein array panel of 124 autoantigens, including nuclear antigens, cytosolic/matrix antigens, and tissue/organ-specific antigens. Data analysis included the following pre-processing steps: (1) background subtraction and averaging of duplicated spots; (2) normalization of the signal intensity of each Ag using internal controls across all samples; and (3) normalized signal intensity (NSI) for each Ag (Ab) completed per sample. NSI files were processed for downstream analysis using the Cluster and Treeview algorithm. Heatmaps were generated using Genesis cluster analysis of microarray data.

### CyTOF

Cryopreserved PBMCs were thawed and stained with a panel of 40 antibodies (Table 2, metal isotope-labeled conjugates, Maxpar Direct Immune Profiling Assay Panel by Fluidigm). Cells were analyzed on a Helios mass cytometer (Fluidigm). Data were normalized and analyzed with gating on CD45+ cells using a cloud-based computational platform OMIQ.ai (Omiq, Inc. Santa Clara, CA). Equal sampling of 7,669 events per sample (lowest common denominator across samples) and a total of 176,387 events across all 23 samples were analyzed using UMAP clustering. Distribution and expression characteristics of phenotypic markers were analyzed using EdgR at OMIQ on the abundance of the 41 clusters between the patient and the control samples. Cluster immune phenotypes were identified following standard immunophenotyping for the Human Immunology Project.

The procedure for immunophenotyping human peripheral blood mononuclear cells by mass cytometry can comprise the method as depicted in FIG. 6, and further described in the following steps:
1. Peripheral blood mononuclear cells (PBMCs) were isolated using density gradient centrifugation in Ficoll-Paque PLUS Media following manufacturer's instruction (Fisher Scientific, Waltham, MA). Cells were washed in phosphate buffered saline (PBS) and suspended in freezing medium containing 90% fetal bovine serum+10% dimethylsulfoxide. Cells were cryopreserved in liquid nitrogen.
2. For cell staining, all staining reagents and buffers were purchased from Standard Biotools (San Francisco, CA). Cells were thawed rapidly and washed twice in Cell Staining Buffer.
3. Cells were resuspended in Cell Staining Buffer and added to FcR block and incubated for 10 min. at room temperature.
4. Cells were transferred to the Direct Immune Profiling antibody cocktail.
5. 5 additional antibodies were added to this cocktail: Integrin-a4b7, CCR9, PD-1, PD-L1, and CTLA4. All antibodies are listed in the Antibody Probes Table (Table 2).
6. Cells were mixed and incubated with the antibody cocktail for 30 min. at room temperature.
7. Cells were washed twice in Cell Staining Buffer and fixed with 1.6% PFA/PBS solution.
8. Cells were washed once with Fix-Perm buffer, and then resuspended in 1 ml Fix Perm buffer containing Cell ID intercalator Ir.
9. Prior to analysis, cells were washed in Cell Acquisition Buffer and counted.
10. Cells were resuspended in Cell Acquisition Buffer containing EQ4 beads (for normalization).
11. Cells were run and data were acquired on a Helios mass cytometer.
12. Following acquisition, data were normalized to EQ4 bead run rates and exported as concatenated FCS files for analysis.
13. Data analysis:
   a. Gating and sampling: Data were gated on live and singlets based on DNA intercalation values, and normalization beads were excluded. Equal sampling of ≥7500 events per sample across all samples are counted in a dataset for analysis.
   b. Dimensionality reduction: Data set were analyzed through UMAP with Epochs 200 and random seeds 2914.
   c. Clustering: Specific cell subsets were defined by cluster analysis with the PhenoGraph algorithm with k=20 Clusters are further depicted on UMAP graphs.
   d. Cell subset clustering: cluster immune phenotypes following standard immunophenotyping for the Human Immunology Project based on antigen marker expression on clusters—heatmap.
   e. The abundance of immune cell subsets (percentage of total cell event per sample) among samples were used for comparison and is shown in heatmap. The abundance of immune cell subsets among samples can be used for statistical analysis. The difference of the abundance on immune cell subsets of interest can also be shown in box or violin plots.

TABLE 2
List of antibodies used for mass cytometry

| Probe/Target | Metal Label | Antibody clone |
|---|---|---|
| CD45 | 89Y | HI30 |
| Cell viability Dye | 103Rh | |
| Normalization Bead | 140Ce | |
| CD196_CCR6 | 141Pr | G034E3 |
| b2-microglobulin | 142Nd | 2M2 |
| CD123_IL-3R | 143Nd | 6H6 |
| CD19 | 144Nd | HIB19 |
| CD4 | 145Nd | RPA-T4 |
| CD8a | 146Nd | RPA-T8 |
| CD11c | 147Sm | Bu15 |
| CD16 | 148Nd | 3G8 |
| CD45RO | 149Sm | UCHL1 |
| CD45RA | 150Nd | HI100 |
| CD161 | 151Eu | HP-3G10 |
| CD194_CCR4 | 152Sm | L291H4 |
| CD25_IL-2Ra | 153Eu | BC96 |
| CD27 | 154Sm | O323 |
| CD57 | 155Gd | HNK-1 |
| CD183_CXCR3 | 156Gd | G025H7 |
| CD185_CXCR5 | 158Gd | J252D4 |
| Integrin_a4b7 | 159Tb | HU117 |
| CD28 | 160Gd | CD28.2 |
| CD38 | 161Dy | HB-7 |
| CCR9 | 162Dy | Polyclonal |
| CD56_NCAM | 163Dy | NCAM16.2 |
| TCRgd | 164Dy | B1 |
| PD-1 | 165Ho | EH12.2H7 |
| CD294 | 166Er | BM16 |
| CD197_CCR7 | 167Er | G043H7 |
| CD14 | 168Er | 63D3 |
| CTLA-4 | 169Tm | BNI3 |
| CD3 | 170Er | UCHT1 |
| CD20 | 171Yb | 2H7 |
| CD66b | 172Yb | G10F5 |
| HLA-DR | 173Yb | LN3 |
| IgD | 174Yb | IA6-2 |
| PD-L1 | 175Lu | 29E.3A3 |
| CD127_IL-7Ra | 176Yb | A019D5 |
| DNA Intercalator 1 | 191Ir | |
| DNA Intercalator 2 | 193Ir | |

### Example 1. Clinical Features and Sample Information on the Patients and the Healthy Control

A 56-year-old woman with a 30 pack-year smoking history presented with hemoptysis. She had a history of chronic obstructive pulmonary disease, hypertension, and dyslipidemia, but no autoimmune, vascular, or neurologic disease. Imaging demonstrated a 1.7 cm right middle lobe mass, a sub-centimeter left parietal brain metastasis, and multiple pleural deposits. Biopsy demonstrated poorly differentiated squamous cell carcinoma with programmed death-ligand 1 (PD-L1) staining greater than 50%.

The patient received single-agent pembrolizumab, which was tolerated well and resulted in prolonged disease control. Almost one year after treatment initiation, she developed blurry vision in the left eye without headache or temporal artery tenderness. On exam, she had 20/200 vision in the left eye and 20/20 vision in the right, an afferent pupillary defect, and severe left optic disc edema. Laboratory analysis was remarkable for C-reactive protein (CRP) of 88 mg/L (reference range 55 mg/L). MRI of the orbits showed no enhancement of orbits or optic nerves. Temporal artery biopsy did not demonstrate active or healed arteritis.

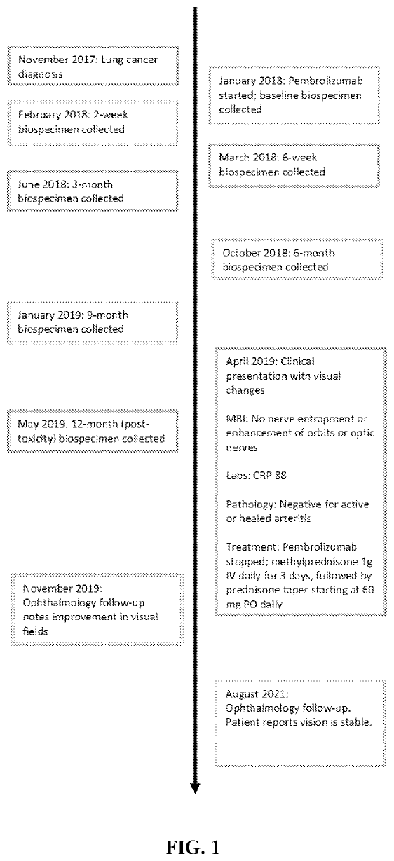

The patient received methylprednisone 1 g IV daily for presumed arteritic ischemic optic neuropathy, with mild subjective improvement in vision, then discharged with a slow prednisone taper starting at 60 mg orally daily. More than one year after the event, the patient remained off any systemic cancer therapy with ongoing disease control but only modest improvement in visual acuity (FIG. 1).

### Example 2: Comparison of Cytokine and Autoantibody Profiles

Cytokine levels were examined in the patient and compared to no toxicity and healthy controls. FIG. 2 displays serial cytokine (FIG. 2A & FIG. 2B) and autoantibody (FIG. 2C & FIG. 2D) determinations. In general, levels of selected cytokines demonstrated modest increases at the 3- and 6-month time-points, but substantial changes did not occur until nine months after ICI initiation. In particular, pro-inflammatory cytokines and chemokines such as IL-6, IL-1β, TNFα, IFNγ, CXCL2, and CCL17 exhibited increased levels at that time-point in the patient but not in the no toxicity (NT) patients treated with ICI. The longitudinal stability of these parameters has been previously demonstrated in the healthy control patients. Similarly, autoantibody levels broadly increased after initiation of ICI. Antibodies against angiotensin receptor (AGTR), α-actin, U1-snRNP A, amyloid, and collagen II exhibited the greatest increases in the patient in comparison to the NT cases. Following initiation of high-dose corticosteroids (12-month time-point), levels of cytokines and autoantibodies broadly and promptly decreased. In summary, these results are consistent with the clinical observation with the onset of irAE of ocular toxicity being accompanied with increased plasma inflammatory cytokines and autoantibodies.

### Example 3: Comparison of Immune Cell Profiles

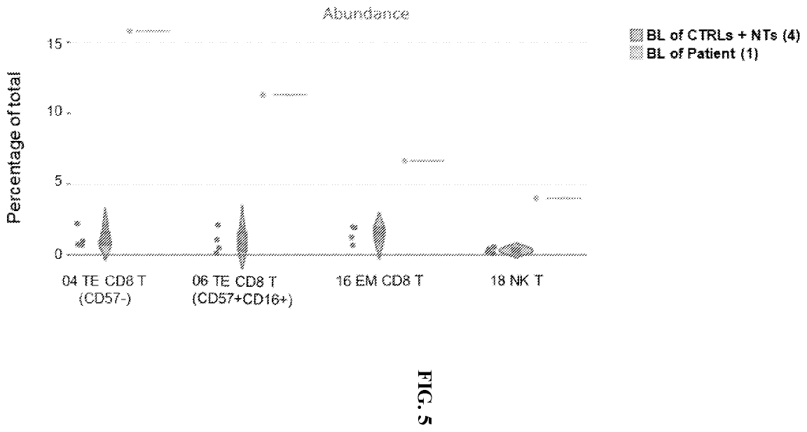

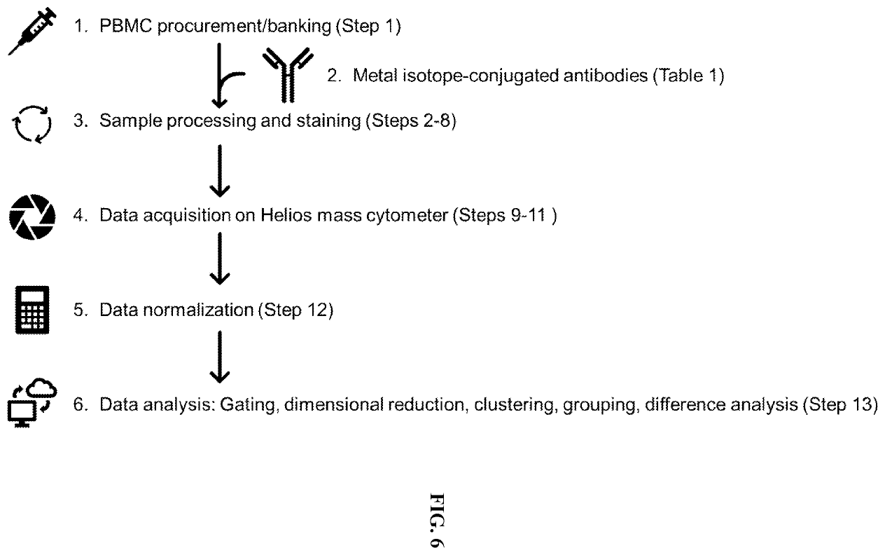

Longitudinal immune cell profiling were compared in peripheral blood samples from the patient (7 time-points), two NT cases (7 time-points), and two healthy controls (CTRL, 2 time-points) using high-dimensional mass cytometry (CyTOF). Based on distinct marker expression, 41 clusters representing B cells, CD4/CD8 T cells, monocytes, and NK cells were identified (FIG. 3A & FIG. 3B). 13 distinctive clusters were identified as having statistically significance (q<0.01) between patient and control samples (FIG. 3C & FIG. 3D) and the immune cell phenotype were identified based on antigen expression (FIG. 3B) and summarized in Table 3. Compared to healthy controls, cancer patients had significantly lower naïve and memory B cells, as well as CD57hi CD56+TE CD8 T cells (FIG. 3D & FIG. 3E). Compared with the controls of no toxicity (NT) cases and healthy subjects, the patient had significantly lower naïve CD4, CD8, and gamma-delta T cells (FIG. 3D, FIG. 3F, & FIG. 4). At later time-points (3, 6, and 9 months; FIG. 3D), increased subsets of effector memory and terminal effector CD8 T cells, and plasmablasts, were exhibited in the patient compared to the NT cases and healthy controls. The disclosed results suggest that higher immune cell subsets of TE CD8 T cells (cluster UP.06 and UP.04), EM CD8 T cells (UP.16), and CD57lo CD56+TE CD8 T cells (UP.18) could be biomarkers to predict ocular toxicity prior to ICI therapy (FIG. 5 & Table 4). Together with plasmablasts (UP.31 and UP.36), CLTA-4+ naïve B cells (UP.39) were associated with the inflammatory response to ocular toxicity (FIG. 3D, shown in red). In summary, the disclosed results identified cellular and humoral immunity as biomarkers in patients who developed irAE after receiving ICI.

TABLE 3
Immune subsets with significant changes between the patient and the controls

| No. | Cluster# | Immune cell phenotype | Expression of antigen markers |
|---|---|---|---|
| 1 | UP.06 | CD57hiCD56− TE CD8 T cells | CD3+CD8+CCR7−CD45RA+CD45RO−CD57hiCD56− |
| 2 | UP.04 | CD57loCD56− TE CD8 T cells | CD3+CD8+CCR7−CD45RA+CD45RO−CD57loCD56− |
| 3 | UP.02 | naïve B cells | CD3−CD19+CD20+IgDhiCD27− |
| 4 | UP.03 | naïve CD4 T cells | CD3+CD4+CCR7+CD45RA+CD45RO− |
| 5 | UP.11 | CD57hiCD56+ TE CD8 T cells | CD3+CD8+CCR7−CD45RA+CD45RO−CD57hiCD56+CD161− |
| 6 | UP.09 | memory B cells | CD3−CD19+CD20+IgDloCD27lo |
| 7 | UP.16 | EM CD8 T cells | CD3+CD8+CCR7−CD45RA−CD45RO+ |
| 8 | UP.18 | CD57loCD56+ TE CD8 T cells | CD3+CD8+CCR7−CD45RA+CD45RO−CD57loCD56+CD161− |
| 9 | UP.13 | naïve CD8 T cells | CD3+CD8+CCR7+CD45RA+CD45RO− |
| 10 | UP.26 | gd T cells | CD3+CD4−CD8−TCRgd+CD45RO+CD27+ |
| 11 | UP.31 | pDC | CD45loCD3−CD19−CD14+HLA-DR+CD123+CXCR3+ |
| 12 | UP.36 | plasmablasts | CD3−CD19+CD20−CD38+ |
| 13 | UP.39 | CTLA-4+ naïve B cells | CD3−CD19+CD20+CD27−IgD+CTLA-4+ |

TABLE 4
Comparison of clusters between the patient
and the controls at baseline

| | UP.04 | UP.06 | UP.16 | UP.18 |
|---|---|---|---|---|
| | CD57loCD56− | CD57hiCD56− | EM CD8 | CD57loCD56+ |
| | TE CD8 T cells | TE CD8 T cells | T cells | TE CD8 T cells |
| Patient | 15.8% | 11.3% | 6.6% | 4.0% |
| NT + | 1.2% | 1.0% | 1.5% | 0.3% |
| CTRL | | | | |
| controls | | | | |

Percentage of cluster in total cell events is shown.

### SUMMARY OF EXAMPLES

Despite years of research and ICI clinical trials, the optimal approach to monitoring and treating irAE remains unknown. Ocular toxicity, which developed in the presented case almost one year after ICI initiation, is an established but rare (<1%) event associated with ICI. The blood-retinal barrier, lack of efferent lymphatics, and upregulation of cytokines such as Fas-L and TNFβ render the orbit an immune privileged structure. Among these cases, uveitis, episcleritis, and blepharitis are the most common manifestations. The results disclosed herein represents the first description of apparent ICI-associated arteritic ischemic optic neuropathy. While late irAE occur less frequently than early toxicities, they are relatively common in long-term responders such as the patient described here.

Analysis of systemic immune parameters revealed a broad but modest increase shortly after ICI initiation. A more profound change occurred nine months after treatment initiation, an event that may explain the delayed-onset irAE. Among cytokines, IL6, IL1β, TNFα, CXCL2, IFNγ, and CCL17, which are involved in the pro-inflammatory cascade, demonstrated increased levels. Prior to onset of patient's visual symptoms, numerous antibodies increased substantially, including AGTR, which is expressed on endothelial cells and pericytes, is associated with vasoconstriction, and implicated in neurovascular ophthalmic disorders.

Accompanying these cytokine and autoantibody derangements, significant increases were observed in terminal effector CD8 T cells, plasmablasts, and CD14+B cells in the patient compared to the NT and healthy controls, particularly after 3 months of ICI therapy. Immune cell populations were relatively stable over time in the healthy and NT controls. Additionally, it was noted that—compared to healthy controls—the cancer patients in this analysis were deficient in both CD8+ T cells and B cells.

The treatment of irAE is largely empirical. For clinically significant events, expert guidelines often recommend initiating relatively high-dose corticosteroids with slow tapers. Despite this aggressive (and potentially toxic) approach, some patients have refractory or recurrent irAE, eventually requiring alternative immunosuppressive agents. Conversely, other patients might benefit from far lower doses and shorter tapers. Laboratory parameters indicating near-term physiologic effects in patients receiving steroids for irAE treatment might allow a more personalized approach to toxicity management. With serial biospecimens from before and after steroid initiation in the present case, observation of immediate and profound suppression across all immune parameters were able to be undertaken. Determining whether such changes correlate with the degree and duration of clinical benefit will assist clinical decision for cancer therapy.

In summary, disclosed herein through an assessment of longitudinal biospecimens, serial autoantibodies, cytokines, and cellular populations were analyzed. Months after ICI initiation and preceding clinical toxicity, a broad increases in cytokines (most notably IL-6, IFN-γ, CXCL2, and CCL-17), autoantibodies (including anti-angiotensin receptor [AGTR], α-actin, and amyloid), CD8 T-cells, and plasmablasts was increased. These changes were not observed in healthy controls and IC-treated patients without irAE. Administration of corticosteroids resulted in immediate and profound decreases in cytokines, autoantibodies, and inflammatory cells, showing a potential for late-onset changes in humoral and cellular immunity in patients receiving ICI and demonstrated the biologic effects of corticosteroids. The humoral and cellular immune biomarkers identified here can be applied for toxicity monitoring and management.

1. A method of predicting and/or monitoring the risk of developing and/or diagnosing immune-related adverse events (irAE) associated with immune checkpoint inhibitor (ICI) treatment in a subject comprising:
a) providing a sample from the subject;
b) assessing immune cell subset in the sample, wherein the assessment comprises determining the abundance of one or more immune cell subsets in the sample; and
c) predicting risk for developing/diagnosing irAE in the subject wherein, the subject is predicted as having a high risk of developing or diagnosed with having irAE if the abundance of one or more immune cell subsets CD57hi CD56− TE CD8 T cells, CD57lo CD56− TE CD8 T cells, naïve B cells, naïve CD4 T cells, CD57hi CD56+TE CD8 T cells, memory B cells, EM CD8 T cells, CD57lo CD56+TE CD8 T cell, naïve CD8 T cells, gamma delta T cells, pDC, plasmablasts, CTLA-4+ naïve B cells, or any combination thereof are different in the subject when compared to the abundance of the one or more immune cell subsets in a control sample.

2. (canceled)

3. The method of claim 1, wherein the irAE comprises ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof.

4. The method of claim 1, wherein the assessment of immune cells is performed before ICI treatment to predict the subject's risk of developing irAE.

5. The method of claim 1, wherein the assessment of immune cells is performed during ICI treatment to diagnose the subject's risk of developing irAE.

6. The method of claim 1, wherein the assessment is performed after ICI treatment to monitor the subject's risk for developing irAE or the subject's recovery from irAE.

7. The method of claim 1, wherein the subject is predicted as high risk of developing irAE if the abundance of one or more of naïve B cells, CD57hiCD56+TE CD8 T cells, memory B cells, gamma delta T cells, naïve CD8 T cells, naïve CD4 T cells, or any combination thereof prior to ICI treatment (baseline) or during ICI treatment are lower in the sample from the subject compared to the abundance in a control sample, and/or if the abundance of one or more of CD57lo CD56− terminal effector (TE) CD8 cells, CD57hi CD56-CD16+TE CD8 T cells, CD57lo CD56+TE CD8 T cells, effector memory (EM) CD8 T cells, or any combination thereof prior to ICI treatment (baseline) are elevated in the sample from the subject compared to the abundance in a control sample.

8. (canceled)

9. The method of claim 1, wherein the subject is diagnosed with irAE if the abundance of one or more of CD57lo CD56− terminal effector CD8 cells, CD57hi CD56− terminal effector CD8 T cells, EM CD8 T cells, plasmacytoid dendritic cells (pDCs), plasmablasts, CLTA-4+ naïve B cells, or any combination thereof are elevated during ICI treatment, compared to the abundance in a control sample.

10. The method of claim 1, wherein the sample is a whole blood, serum, plasma, cerebrospinal fluid, pleural fluid, pericardial fluid, peritoneal fluid, bone marrow, or tissue, urine, cerebrospinal fluid (CSF), or other body fluid.

11. The method of claim 1, wherein said ICI treatment is administered as part of a cancer treatment.

12. The method of claim 9, wherein said ICI treatment comprises administration of an inhibitor of PD-1, PD-L1, TIM-3, LAG-3, CTLA-4, CSF-1R, or any combination thereof.

13. The method of claim 1, wherein assessing the immune cells (step b) comprises mass cytometry (cytometry by time-of-flight CyTOF), immune profiling assay, flow cytometry, fluorescence-activated cell sorting (FACS), or immunomagnetic separation.

14. The method of claim 1, wherein the method further comprises assessing the level of an autoantibody in the subject, wherein the autoantibody comprises one or more of angiotensin receptor (AGTR), α-actin, U1-snRNP A, amyloid, collagen II, or any combination thereof; wherein the assessing comprises identifying if the level of one or more autoantibodies are elevated in the sample from the subject compared to the level in a control sample.

15. (canceled)

16. The method of claim 1, wherein the method further comprises assessing the expression of a cytokine in the subject, wherein the cytokine is selected from one or more of IL-6, IL-1β, TNFα, IFNγ, CXCL2, CCL17, or any combination thereof; wherein the assessing comprises identifying if expression of one or more cytokines are elevated in the sample from the subject compared to the expression in a control sample.

17. (canceled)

18. The method of claim 1, further comprising repeating steps (a)-(c) at a second time point, thereby permitting determination of a change in the subject's risk of developing irAE and/or diagnosis of irAE in the subject.

19. The method of claim 1, further comprising predicting the subject as having low risk if (i) the abundance of one or more of naïve B cells, CD57hiCD56+TE CD8 T cells, memory B cells, gamma delta T cells, naïve CD8 T cells, naïve CD4 T cells, or any combination thereof prior to ICI treatment (baseline) or during ICI treatment are higher in the sample from the subject compared to the abundance in a control sample; (ii) the abundance of one or more of CD57lo CD56− TE CD8 cells, CD57hi CD56− TE CD8 T cells, effector memory (EM) CD8 T cells, CD57lo CD56+TE CD8 T cells, or any combination thereof prior to ICI treatment (baseline) are lower or equivalent compared to the abundance in a control sample; and/or (iii) if the abundance of one or more of CD57lo CD56− TE CD8 cells, CD57hi CD56− TE CD8 T cells, EM CD8 T cells, pDCs, plasmablasts, CLTA-4+ naïve B cells, or any combination thereof are lower or equivalent in the sample from the subject, during ICI treatment, compared to the abundance in a control sample.

20. The method of claim 18, further comprising treating the subject with an ICI therapy when the subject is predicted to have a low risk for developing irAE.

21. The method of claim 1, further comprising treating the subject predicted as having a high risk of developing irAE with a non-ICI therapy or treating said subject with an ICI therapy and an irAE mitigating therapy, wherein the irAE mitigating therapy comprises corticosteroids (e.g., prednisone, methylprednisolone, dexamethasone, budesonide), TNF inhibitors (e.g., infliximab), hormone replacement (e.g., hydrocortisone, levothyroxine), BRAF inhibitor (e.g. vemurafenib, dabrafenib, and encorafenib), MEK inhibitor (e.g. binimetinib, cobimetinib, selumetinib, trametinib), CXCL8 inhibitors (e.g. repertaxin), or any combination thereof.

22. A method of treating a subject with cancer comprising:
(a) providing a sample from the subject;
(b) assessing immune cell subset in the sample, wherein the assessment comprises determining the abundance of one or more of immune cell subsets in CD57hi CD56− TE CD8 T cells, CD57lo CD56− TE CD8 T cells, naïve B cells, naïve CD4 T cells, CD57hi CD56+TE CD8 T cells, memory B cells, EM CD8 T cells, CD57lo CD56+TE CD8 T cell, naïve CD8 T cells, gamma delta T cells, pDC, plasmablasts, CTLA-4+ naïve B cells, or any combination thereof in the sample;
(c) diagnosing the subject's risk of developing irAE, wherein the subject is diagnosed as:
(i) high risk when the abundance of one or more of naïve B cells, CD57hiCD56+TE CD8 T cells, memory B cells, gamma delta T cells, naïve CD8 T cells, naïve CD4 T cells, or any combination thereof are lower in the sample from the subject compared to the abundance in a control sample;
(ii) low risk of developing irAE if the abundance of one or more of naïve B cells, CD57hiCD56+TE CD8 T cells, memory B cells, gamma delta T cells, naïve CD8 T cells, naïve CD4 T cells, or any combination thereof elevated in the sample from the subject compared to the abundance in a control sample;
(ii) low risk when the abundance of one or more of CD57lo CD56− terminal effector CD8 cells, CD57hi CD56-terminal effector CD8 T cells, EM CD8 T cells, plasmacytoid dendritic cells, plasmablasts, CLTA-4+ naïve B cells, or any combination thereof are lower or equal in the sample from the subject when compared to the abundance in a control sample; and/or
(iii) high risk when the abundance of one or more of CD57lo CD56− terminal effector CD8 cells, CD57hi CD56-terminal effector CD8 T cells, EM CD8 T cells, plasmacytoid dendritic cells, plasmablasts, CLTA-4+ naïve B cells, or any combination thereof are greater in the sample from the subject when compared to abundance in a control sample;
(d) treating the subject with:
(i) an ICI therapy if subject is diagnosed as low risk of developing irAE;
(ii) a non-ICI therapy if the subject is diagnosed as high risk of developing irAE; or
(iii) an ICI therapy and an irAE mitigating therapy if the subject is diagnosed as high risk of developing irAE.

23. The method of claim 22, wherein irAE comprises ocular toxicity, rash, dermatitis, pruritus, colitis, hepatitis, nephritis, arthritis, myositis, myocarditis, pneumonitis, thyroiditis, hypophysitis, adrenalitis, gastritis, pancreatitis, vasculitis, diabetes, myasthenia gravis, encephalitis, peripheral neuropathy, meningitis, hemolytic anemia, thrombocytopenia, hemophagocytic lymphohistiocytosis/macrophage activation syndrome (HLH/MAS), aplastic anemia, pure red cell aplasia, neutropenia, or any combination thereof.

24. The method of claim 22, wherein the assessment further comprises detecting expression of a cytokine in the sample; and identifying if expression of one or more of cytokines are elevated in the sample from the subject compared to the expression in a control sample; wherein the cytokine comprises one or more of IL-6, IL-1β, TNFα, IFNγ, CXCL2, CCL17, or any combination thereof.

25. (canceled)

26. The method of claim 22, wherein the assessment further comprises detecting the level of an autoantibody in the sample; and identifying if the level of one or more autoantibodies are elevated in the sample from the subject compared to the level in a control sample; wherein the autoantibody comprises one or more of angiotensin receptor (AGTR), α-actin, U1-snRNP A, amyloid, collagen II, or any combination thereof.

27. (canceled)

28. The method of claim 22, wherein said ICI therapy comprises administration of an inhibitor of PD-1, PD-L1, TIM-3, LAG-3, CTLA-4, CSF-1R, or any combination thereof.

29. A method of identifying the presence of at least one differentially expressed immune cell subset associated with irAE in a biological sample of a subject with cancer, the method comprising:
a. providing a biological sample from the subject;
b. assessing immune cells in the sample, wherein the assessment comprises detecting the abundance of one or more immune cell subsets disclosed in CD57hi CD56− TE CD8 T cells, CD57lo CD56− TE CD8 T cells, naïve B cells, naïve CD4 T cells, CD57hi CD56+TE CD8 T cells, memory B cells, EM CD8 T cells, CD57lo CD56+TE CD8 T cell, naïve CD8 T cells, gamma delta T cells, pDC, plasmablasts, CTLA-4+ naïve B cells, or any combination thereof; and
c. comparing the abundance of one or more immune cell subsets to the abundance in a control sample to identify the presence of differentially expressed immune cell subset(s);
wherein the subject is planning, undergoing, or has completed immune checkpoint inhibitor (ICI) treatment.

30. (canceled)

31. (canceled)

32. (canceled)

33. (canceled)

34. (canceled)

35. (canceled)

36. The method of claim 29,
wherein the assessment comprises determining a baseline or a pre-treatment profile that correlates with future toxicity; or the assessment comprises an interim profile that correlates with ICI treatment before the onset of irAE: or the assessment comprises a profile that correlates with onset of irAE during treatment; and
wherein the baseline or a pre-treatment profile, the interim profile and/or the profile that correlates with the onset of irAE during treatments comprises assessment of one or more of CD57lo CD56− terminal effector TE CD8 cells, CD57hi CD56− terminal effector CD8 T cells, Effector memory (EM) EM CD8 T cells, NK T Plasmacytoid dendritic cells (pDCs), or any combination thereof, and/or naïve B cells, CD57hiCD56+TE CD8 T cells, memory B cells, gamma delta T cells, naïve CD8 T cells, naïve CD4 T cells, or any combination thereof

37. (canceled)

38. (canceled)

39. (canceled)

40. (canceled)

41. (canceled)
